SEQUENCE LISTING (93 sequences)

SEQ ID NO: 1 - protein, 162 aa, Mus musculus
CDLPHTYNLGNKRALTVLEEMRRLPPLSCLKDRKDFGFPLEKVDNQQIQKAQAILVLRDLTQQILNLFTSKDLSATWNATLLDSFCNDLHQQLNDLKACVMQEPPLTQEDSLLAVRTYFHRITVYLRKKKHSLCAWEVIRAEVWRALSSSTNLLARLSEEKE

SEQ ID NO: 2 - protein, 233 aa, Homo sapiens
EPKSCDKTHTCPPCPAPELLGGPSVFLFPPKPKDQLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLYSKLTVDKSRWQQGNVFSCSVLHEALHNHYTQKSLSLSPGKHV

SEQ ID NO: 3 - protein, 13 aa, Homo sapiens
AKFVAAWTLKAAA

SEQ ID NO: 4 - protein, 15 aa, synthetic construct
  REGION 1..15: note = artificially synthesized sequence
GGGGSGGGGSGGGGS

SEQ ID NO: 5 - protein, 24 aa, Homo sapiens
MARLCAFLMILVMMSYYWSACSLG

SEQ ID NO: 6 - protein, 119 aa, Hepatitis B virus
MGGWSSKPRKGMGTNLSVPNPLGFFPDHQLDPAFGANSNNPDWDFNPIKDHWPAANQVGVGAFGPGLTPPHGGILGWSPQAQGILTTVSTIPPPASTNRQSGRQPTPISPPLRDSHPQA

SEQ ID NO: 7 - protein, 226 aa, Hepatitis B virus
MENTTSGFLGPLLVLQAGFFLLTRILTIPQSLDSWWTSLNFLGGAPTCPGQNSQSPTSNHSPTSCPPICPGYRWMCLRRFIIFLFILLLCLIFLLVLLDYQGMLPVCPLLPGTSTTSTGPCKTCTIPAQGTSMFPSCCCTKPSDGNCTCIPIPSSWAFARFLWEWASVRFSWLSLLVPFVQWFVGLSPTVWLSVIWMMWYWGPSLYNILSPFLPLLPIFFCLWVYI

SEQ ID NO: 8 - protein, 223 aa, Hepatitis B virus
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 9 - protein, 326 aa, Hepatitis B virus
DTICIGYHANNSTDTVDTVLEKNVTVTHSVNLLEDSHNGKLCRLKGIAPLQLGKCNIAGWLLGNPECDPLLPVRSWSYIVETPNSENGICYPGDFIDYEELREQLSSVSSFERFEIFPKESSWPNHNTNGVTAACSHEGKSSFYRNLLWLTEKEGSYPKLKNSYVNKKGKEVLVLWGIHHPPNSKEQQNLYQNENAYVSVVTSNYNRRFTPEIAERPKVRDQAGRMNYYWTLLKPGDTIIFEANGNLIAPMYAFALSRGFGSGIITSNASMHECNTKCQTPLGAINSSLPYQNIHPVTIGECPKYVRSAKLRMVTGLRNNPSIQSR

SEQ ID NO: 10 - protein, 98 aa, Hepatitis B virus
MHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQKP

SEQ ID NO: 11 - protein, 527 aa, Mus musculus
CDLPHTYNLGNKRALTVLEEMRRLPPLSCLKDRKDFGFPLEKVDNQQIQKAQAILVLRDLTQQILNLFTSKDLSATWNATLLDSFCNDLHQQLNDLKACVMQEPPLTQEDSLLAVRTYFHRITVYLRKKKHSLCAWEVIRAEVWRALSSSTNLLARLSEEKESGGGGSGGGGSGGGGSGGGGRTMGQNLSTSNPLGFFPDHQLDPAFRANTANPDWDFNPNKDTWPDANKVGAGAFGLGFTPPHGGLLGWSPQAQGILQTLPANPPPASTNRQTGRQPTPLSPPLRNTHPQAFEEPKSCDKTHTCPPCPAPELLGGPSVFLFPPKPKDQLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLYSKLTVDKSRWQQGNVFSCSVLHEALHNHYTQKSLSLSPGKHV

SEQ ID NO: 12 - protein, 642 aa, Mus musculus
CDLPHTYNLGNKRALTVLEEMRRLPPLSCLKDRKDFGFPLEKVDNQQIQKAQAILVLRDLTQQILNLFTSKDLSATWNATLLDSFCNDLHQQLNDLKACVMQEPPLTQEDSLLAVRTYFHRITVYLRKKKHSLCAWEVIRAEVWRALSSSTNLLARLSEEKESGGGGSGGGGSGGGGSGGGGRTRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFFEEPKSCDKTHTCPPCPAPELLGGPSVFLFPPKPKDQLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLYSKLTVDKSRWQQGNVFSCSVLHEALHNHYTQKSLSLSPGKHV

SEQ ID NO: 13 - protein, 745 aa, Mus musculus
CDLPHTYNLGNKRALTVLEEMRRLPPLSCLKDRKDFGFPLEKVDNQQIQKAQAILVLRDLTQQILNLFTSKDLSATWNATLLDSFCNDLHQQLNDLKACVMQEPPLTQEDSLLAVRTYFHRITVYLRKKKHSLCAWEVIRAEVWRALSSSTNLLARLSEEKESGGGGSGGGGSGGGGSGGGGRTDTICIGYHANNSTDTVDTVLEKNVTVTHSVNLLEDSHNGKLCRLKGIAPLQLGKCNIAGWLLGNPECDPLLPVRSWSYIVETPNSENGICYPGDFIDYEELREQLSSVSSFERFEIFPKESSWPNHNTNGVTAACSHEGKSSFYRNLLWLTEKEGSYPKLKNSYVNKKGKEVLVLWGIHHPPNSKEQQNLYQNENAYVSVVTSNYNRRFTPEIAERPKVRDQAGRMNYYWTLLKPGDTIIFEANGNLIAPMYAFALSRGFGSGIITSNASMHECNTKCQTPLGAINSSLPYQNIHPVTIGECPKYVRSAKLRMVTGLRNNPSIQSRFEEPKSCDKTHTCPPCPAPELLGGPSVFLFPPKPKDQLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLYSKLTVDKSRWQQGNVFSCSVLHEALHNHYTQKSLSLSPGKHV

SEQ ID NO: 14 - protein, 517 aa, Mus musculus
CDLPHTYNLGNKRALTVLEEMRRLPPLSCLKDRKDFGFPLEKVDNQQIQKAQAILVLRDLTQQILNLFTSKDLSATWNATLLDSFCNDLHQQLNDLKACVMQEPPLTQEDSLLAVRTYFHRITVYLRKKKHSLCAWEVIRAEVWRALSSSTNLLARLSEEKESGGGGSGGGGSGGGGSGGGGRTMHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQKPFEEPKSCDKTHTCPPCPAPELLGGPSVFLFPPKPKDQLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLYSKLTVDKSRWQQGNVFSCSVLHEALHNHYTQKSLSLSPGKHV

SEQ ID NO: 15 - protein, 411 aa, Mus musculus
CDLPHTYNLGNKRALTVLEEMRRLPPLSCLKDRKDFGFPLEKVDNQQIQKAQAILVLRDLTQQILNLFTSKDLSATWNATLLDSFCNDLHQQLNDLKACVMQEPPLTQEDSLLAVRTYFHRITVYLRKKKHSLCAWEVIRAEVWRALSSSTNLLARLSEEKESGGGGSGGGGSGGGGSGGGGRTDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 16 - protein, 337 aa, Mus musculus
MGQNLSTSNPLGFFPDHQLDPAFRANTANPDWDFNPNKDTWPDANKVGAGAFGLGFTPPHGGLLGWSPQAQGILQTLPANPPPASTNRQTGRQPTPLSPPLRNTHPQAFEDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 452 aa, Mus musculus
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFFEDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 555 aa, Mus musculus
DTICIGYHANNSTDTVDTVLEKNVTVTHSVNLLEDSHNGKLCRLKGIAPLQLGKCNIAGWLLGNPECDPLLPVRSWSYIVETPNSENGICYPGDFIDYEELREQLSSVSSFERFEIFPKESSWPNHNTNGVTAACSHEGKSSFYRNLLWLTEKEGSYPKLKNSYVNKKGKEVLVLWGIHHPPNSKEQQNLYQNENAYVSVVTSNYNRRFTPEIAERPKVRDQAGRMNYYWTLLKPGDTIIFEANGNLIAPMYAFALSRGFGSGIITSNASMHECNTKCQTPLGAINSSLPYQNIHPVTIGECPKYVRSAKLRMVTGLRNNPSIQSRFEDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 327 aa, Mus musculus
MHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQKPFEDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 241 aa, Homo sapiens
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRGGGGSGGGGSGGGGSEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSA

SEQ ID NO: 21 - protein, 165 aa, Homo sapiens [sequence omitted]

SEQ ID NO: 22 - protein, 165 aa, Homo sapiens
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFRRITLYLKEKKYSPCAWEVVRAEIMRSFSLSTNLQESLRSKE

SEQ ID NO: 23 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 24 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 6 aa, synthetic construct
  REGION 1..6: note = artificially synthesized sequence
GSGSGS

SEQ ID NO: 26 - protein, 108 aa, Hepatitis B virus
MGQNLSTSNPLGFFPDHQLDPAFRANTANPDWDFNPNKDTWPDANKVGAGAFGLGFTPPHGGLLGWSPQAQGILQTLPANPPPASTNRQTGRQPTPLSPPLRNTHPQA

SEQ ID NO: 27 - protein, 226 aa, Hepatitis B virus
MENITSGLLGPLLVLQAGFFLLTRILTIPQSLDSWWTSLSFLGEAPVCLGQNSQSPTRNHSPTSCPPICPGYRWMCLRRFIIFLFILLLCLIFLLVLLDYQGMLPVCPLIPGSTTTSTGPCKTCTTPAQGNSMFPSCCCTKPTDGNCTCIPIPSSWAFAKYLWEWASVRFSWLSLLVPFVQWFVGLSPTVWLSAIWMIWYWGPSLYSIVCPFTPLLQIFCCLWVFI

SEQ ID NO: 28 - protein, 226 aa, Hepatitis B virus
MENITSGFLGPLLVLQAGFFLLTRILTIPQSLDSWWTSLNFLGGTTVCLGQSSQSPTSNHSPTSCPPTCPGYRWMCLRRFIIFLFILLLCLIFLLVLLDYQGMLPVCPLIPGSSTTSTGPCRTCMTTAQGTSMYPSCCCTKPSDGNCTCIPIPSSWAFGKFLWEWASARFSWLSLLVPFVQWFVGLSPTVWLSVIWMMWYWGPSLYSILSPFLPLLPIFFCLWVYI

SEQ ID NO: 29 - protein, 534 aa, Mus musculus
CDLPHTYNLGNKRALTVLEEMRRLPPLSCLKDRKDFGFPLEKVDNQQIQKAQAILVLRDLTQQILNLFTSKDLSATWNATLLDSFCNDLHQQLNDLKACVMQEPPLTQEDSLLAVRTYFHRITVYLRKKKHSLCAWEVIRAEVWRALSSSTNLLARLSEEKEGGGGSGGGGSGGGGSRTAKFVAAWTLKAAAGSGSGSMGQNLSTSNPLGFFPDHQLDPAFRANTANPDWDFNPNKDTWPDANKVGAGAFGLGFTPPHGGLLGWSPQAQGILQTLPANPPPASTNRQTGRQPTPLSPPLRNTHPQAFEDKTHTCPPCPAPELLGGPSVFLFPPKPKDQLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLYSKLTVDKSRWQQGNVFSCSVLHEALHNHYTQKSLSLSPGK

SEQ ID NO: 30 - protein, 649 aa, Mus musculus
CDLPHTYNLGNKRALTVLEEMRRLPPLSCLKDRKDFGFPLEKVDNQQIQKAQAILVLRDLTQQILNLFTSKDLSATWNATLLDSFCNDLHQQLNDLKACVMQEPPLTQEDSLLAVRTYFHRITVYLRKKKHSLCAWEVIRAEVWRALSSSTNLLARLSEEKEGGGGSGGGGSGGGGSRTAKFVAAWTLKAAAGSGSGSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFFEDKTHTCPPCPAPELLGGPSVFLFPPKPKDQLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLYSKLTVDKSRWQQGNVFSCSVLHEALHNHYTQKSLSLSPGK

SEQ ID NO: 31 - protein, 752 aa, Mus musculus
CDLPHTYNLGNKRALTVLEEMRRLPPLSCLKDRKDFGFPLEKVDNQQIQKAQAILVLRDLTQQILNLFTSKDLSATWNATLLDSFCNDLHQQLNDLKACVMQEPPLTQEDSLLAVRTYFHRITVYLRKKKHSLCAWEVIRAEVWRALSSSTNLLARLSEEKEGGGGSGGGGSGGGGSRTAKFVAAWTLKAAAGSGSGSDTICIGYHANNSTDTVDTVLEKNVTVTHSVNLLEDSHNGKLCRLKGIAPLQLGKCNIAGWLLGNPECDPLLPVRSWSYIVETPNSENGICYPGDFIDYEELREQLSSVSSFERFEIFPKESSWPNHNTNGVTAACSHEGKSSFYRNLLWLTEKEGSYPKLKNSYVNKKGKEVLVLWGIHHPPNSKEQQNLYQNENAYVSVVTSNYNRRFTPEIAERPKVRDQAGRMNYYWTLLKPGDTIIFEANGNLIAPMYAFALSRGFGSGIITSNASMHECNTKCQTPLGAINSSLPYQNIHPVTIGECPKYVRSAKLRMVTGLRNNPSIQSRFEDKTHTCPPCPAPELLGGPSVFLFPPKPKDQLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLYSKLTVDKSRWQQGNVFSCSVLHEALHNHYTQKSLSLSPGK

SEQ ID NO: 32 - protein, 524 aa, Mus musculus
CDLPHTYNLGNKRALTVLEEMRRLPPLSCLKDRKDFGFPLEKVDNQQIQKAQAILVLRDLTQQILNLFTSKDLSATWNATLLDSFCNDLHQQLNDLKACVMQEPPLTQEDSLLAVRTYFHRITVYLRKKKHSLCAWEVIRAEVWRALSSSTNLLARLSEEKEGGGGSGGGGSGGGGSRTAKFVAAWTLKAAAGSGSGSMHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQKPFEDKTHTCPPCPAPELLGGPSVFLFPPKPKDQLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLYSKLTVDKSRWQQGNVFSCSVLHEALHNHYTQKSLSLSPGK

SEQ ID NO: 33 - protein, 411 aa, Mus musculus; identical to SEQ ID NO: 15

SEQ ID NO: 34 - protein, 356 aa, Mus musculus
AKFVAAWTLKAAAGSGSGSMGQNLSTSNPLGFFPDHQLDPAFRANTANPDWDFNPNKDTWPDANKVGAGAFGLGFTPPHGGLLGWSPQAQGILQTLPANPPPASTNRQTGRQPTPLSPPLRNTHPQAFEDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 471 aa, Mus musculus
AKFVAAWTLKAAAGSGSGSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFFEDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - protein, 574 aa, Mus musculus
AKFVAAWTLKAAAGSGSGSDTICIGYHANNSTDTVDTVLEKNVTVTHSVNLLEDSHNGKLCRLKGIAPLQLGKCNIAGWLLGNPECDPLLPVRSWSYIVETPNSENGICYPGDFIDYEELREQLSSVSSFERFEIFPKESSWPNHNTNGVTAACSHEGKSSFYRNLLWLTEKEGSYPKLKNSYVNKKGKEVLVLWGIHHPPNSKEQQNLYQNENAYVSVVTSNYNRRFTPEIAERPKVRDQAGRMNYYWTLLKPGDTIIFEANGNLIAPMYAFALSRGFGSGIITSNASMHECNTKCQTPLGAINSSLPYQNIHPVTIGECPKYVRSAKLRMVTGLRNNPSIQSRFEDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 37 - protein, 346 aa, Mus musculus
AKFVAAWTLKAAAGSGSGSMHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQKPFEDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - protein, 518 aa, Homo sapiens
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKEGGGGSGGGGSGGGGSRTMGQNLSTSNPLGFFPDHQLDPAFRANTANPDWDFNPNKDTWPDANKVGAGAFGLGFTPPHGGLLGWSPQAQGILQTLPANPPPASTNRQTGRQPTPLSPPLRNTHPQAFEDKTHTCPPCPAPELLGGPSVFLFPPKPKDQLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLYSKLTVDKSRWQQGNVFSCSVLHEALHNHYTQKSLSLSPGK

SEQ ID NO: 39 - protein, 633 aa, Homo sapiens
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKEGGGGSGGGGSGGGGSRTRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFFEDKTHTCPPCPAPELLGGPSVFLFPPKPKDQLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLYSKLTVDKSRWQQGNVFSCSVLHEALHNHYTQKSLSLSPGK

SEQ ID NO: 40 - protein, 736 aa, Homo sapiens
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKEGGGGSGGGGSGGGGSRTDTICIGYHANNSTDTVDTVLEKNVTVTHSVNLLEDSHNGKLCRLKGIAPLQLGKCNIAGWLLGNPECDPLLPVRSWSYIVETPNSENGICYPGDFIDYEELREQLSSVSSFERFEIFPKESSWPNHNTNGVTAACSHEGKSSFYRNLLWLTEKEGSYPKLKNSYVNKKGKEVLVLWGIHHPPNSKEQQNLYQNENAYVSVVTSNYNRRFTPEIAERPKVRDQAGRMNYYWTLLKPGDTIIFEANGNLIAPMYAFALSRGFGSGIITSNASMHECNTKCQTPLGAINSSLPYQNIHPVTIGECPKYVRSAKLRMVTGLRNNPSIQSRFEDKTHTCPPCPAPELLGGPSVFLFPPKPKDQLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLYSKLTVDKSRWQQGNVFSCSVLHEALHNHYTQKSLSLSPGK

SEQ ID NO: 41 - protein, 508 aa, Homo sapiens
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKEGGGGSGGGGSGGGGSRTMHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQKPFEDKTHTCPPCPAPELLGGPSVFLFPPKPKDQLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLYSKLTVDKSRWQQGNVFSCSVLHEALHNHYTQKSLSLSPGK

SEQ ID NO: 42 - protein, 414 aa, Homo sapiens
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKESGGGGSGGGGSGGGGSGGGGRTDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFKLVSKLTVDKSRWQQGNVFSCSVMHEALHNHYTQKSLSLSPGK

SEQ ID NO: 43 - protein, 337 aa, Homo sapiens
MGQNLSTSNPLGFFPDHQLDPAFRANTANPDWDFNPNKDTWPDANKVGAGAFGLGFTPPHGGLLGWSPQAQGILQTLPANPPPASTNRQTGRQPTPLSPPLRNTHPQAFEDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 44 - protein, 452 aa, Homo sapiens; identical to SEQ ID NO: 17

SEQ ID NO: 45 - protein, 555 aa, Homo sapiens; identical to SEQ ID NO: 18

SEQ ID NO: 46 - protein, 327 aa, Homo sapiens; identical to SEQ ID NO: 19

SEQ ID NO: 47 - protein, 520 aa, Homo sapiens
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKEAKFVAAWTLKAAAGSGSGSMGQNLSTSNPLGFFPDHQLDPAFRANTANPDWDFNPNKDTWPDANKVGAGAFGLGFTPPHGGLLGWSPQAQGILQTLPANPPPASTNRQTGRQPTPLSPPLRNTHPQAFEDKTHTCPPCPAPELLGGPSVFLFPPKPKDQLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLYSKLTVDKSRWQQGNVFSCSVLHEALHNHYTQKSLSLSPGK

SEQ ID NO: 48 - protein, 635 aa, Homo sapiens
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKEAKFVAAWTLKAAAGSGSGSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFFEDKTHTCPPCPAPELLGGPSVFLFPPKPKDQLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLYSKLTVDKSRWQQGNVFSCSVLHEALHNHYTQKSLSLSPGK

SEQ ID NO: 49 - protein, 738 aa, Homo sapiens
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKEAKFVAAWTLKAAAGSGSGSDTICIGYHANNSTDTVDTVLEKNVTVTHSVNLLEDSHNGKLCRLKGIAPLQLGKCNIAGWLLGNPECDPLLPVRSWSYIVETPNSENGICYPGDFIDYEELREQLSSVSSFERFEIFPKESSWPNHNTNGVTAACSHEGKSSFYRNLLWLTEKEGSYPKLKNSYVNKKGKEVLVLWGIHHPPNSKEQQNLYQNENAYVSVVTSNYNRRFTPEIAERPKVRDQAGRMNYYWTLLKPGDTIIFEANGNLIAPMYAFALSRGFGSGIITSNASMHECNTKCQTPLGAINSSLPYQNIHPVTIGECPKYVRSAKLRMVTGLRNNPSIQSRFEDKTHTCPPCPAPELLGGPSVFLFPPKPKDQLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLYSKLTVDKSRWQQGNVFSCSVLHEALHNHYTQKSLSLSPGK

SEQ ID NO: 50 - protein, 510 aa, Homo sapiens
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKEAKFVAAWTLKAAAGSGSGSMHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQKPFEDKTHTCPPCPAPELLGGPSVFLFPPKPKDQLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLYSKLTVDKSRWQQGNVFSCSVLHEALHNHYTQKSLSLSPGK

SEQ ID NO: 51 - protein, 414 aa, Homo sapiens
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKESGGGGSGGGGSGGGGSGGGGRTDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 52 - protein, 356 aa, Homo sapiens; identical to SEQ ID NO: 34

SEQ ID NO: 53 - protein, 471 aa, Homo sapiens; identical to SEQ ID NO: 35

SEQ ID NO: 54 - protein, 574 aa, Homo sapiens; identical to SEQ ID NO: 36

SEQ ID NO: 55 - protein, 346 aa, Homo sapiens; identical to SEQ ID NO: 37

SEQ ID NO: 56 - protein, 518 aa, Homo sapiens
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFRRITLYLKEKKYSPCAWEVVRAEIMRSFSLSTNLQESLRSKEGGGGSGGGGSGGGGSRTMGQNLSTSNPLGFFPDHQLDPAFRANTANPDWDFNPNKDTWPDANKVGAGAFGLGFTPPHGGLLGWSPQAQGILQTLPANPPPASTNRQTGRQPTPLSPPLRNTHPQAFEDKTHTCPPCPAPELLGGPSVFLFPPKPKDQLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLYSKLTVDKSRWQQGNVFSCSVLHEALHNHYTQKSLSLSPGK

SEQ ID NO: 57 - protein, 633 aa, Homo sapiens
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFRRITLYLKEKKYSPCAWEVVRAEIMRSFSLSTNLQESLRSKEGGGGSGGGGSGGGGSRTRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFFEDKTHTCPPCPAPELLGGPSVFLFPPKPKDQLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLYSKLTVDKSRWQQGNVFSCSVLHEALHNHYTQKSLSLSPGK

SEQ ID NO: 58 - protein, 736 aa, Homo sapiens
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFRRITLYLKEKKYSPCAWEVVRAEIMRSFSLSTNLQESLRSKEGGGGSGGGGSGGGGSRTDTICIGYHANNSTDTVDTVLEKNVTVTHSVNLLEDSHNGKLCRLKGIAPLQLGKCNIAGWLLGNPECDPLLPVRSWSYIVETPNSENGICYPGDFIDYEELREQLSSVSSFERFEIFPKESSWPNHNTNGVTAACSHEGKSSFYRNLLWLTEKEGSYPKLKNSYVNKKGKEVLVLWGIHHPPNSKEQQNLYQNENAYVSVVTSNYNRRFTPEIAERPKVRDQAGRMNYYWTLLKPGDTIIFEANGNLIAPMYAFALSRGFGSGIITSNASMHECNTKCQTPLGAINSSLPYQNIHPVTIGECPKYVRSAKLRMVTGLRNNPSIQSRFEDKTHTCPPCPAPELLGGPSVFLFPPKPKDQLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLYSKLTVDKSRWQQGNVFSCSVLHEALHNHYTQKSLSLSPGK

SEQ ID NO: 59 - protein, 508 aa, Homo sapiens
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFRRITLYLKEKKYSPCAWEVVRAEIMRSFSLSTNLQESLRSKEGGGGSGGGGSGGGGSRTMHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQKPFEDKTHTCPPCPAPELLGGPSVFLFPPKPKDQLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLYSKLTVDKSRWQQGNVFSCSVLHEALHNHYTQKSLSLSPGK

SEQ ID NO: 60 - protein, 414 aa, Homo sapiens
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFRRITLYLKEKKYSPCAWEVVRAEIMRSFSLSTNLQESLRSKESGGGGSGGGGSGGGGSGGGGRTDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 61 - protein, 337 aa, Homo sapiens; identical to SEQ ID NO: 16

SEQ ID NO: 62 - protein, 452 aa, Homo sapiens; identical to SEQ ID NO: 17

SEQ ID NO: 63 - protein, 555 aa, Homo sapiens; identical to SEQ ID NO: 18

SEQ ID NO: 64 - protein, 327 aa, Homo sapiens; identical to SEQ ID NO: 19

SEQ ID NO: 65 - protein, 520 aa, Homo sapiens
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFRRITLYLKEKKYSPCAWEVVRAEIMRSFSLSTNLQESLRSKEAKFVAAWTLKAAAGSGSGSMGQNLSTSNPLGFFPDHQLDPAFRANTANPDWDFNPNKDTWPDANKVGAGAFGLGFTPPHGGLLGWSPQAQGILQTLPANPPPASTNRQTGRQPTPLSPPLRNTHPQAFEDKTHTCPPCPAPELLGGPSVFLFPPKPKDQLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLYSKLTVDKSRWQQGNVFSCSVLHEALHNHYTQKSLSLSPGK

SEQ ID NO: 66 - protein, 635 aa, Homo sapiens
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFRRITLYLKEKKYSPCAWEVVRAEIMRSFSLSTNLQESLRSKEAKFVAAWTLKAAAGSGSGSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFFEDKTHTCPPCPAPELLGGPSVFLFPPKPKDQLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLYSKLTVDKSRWQQGNVFSCSVLHEALHNHYTQKSLSLSPGK

SEQ ID NO: 67 - protein, 738 aa, Homo sapiens
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFRRITLYLKEKKYSPCAWEVVRAEIMRSFSLSTNLQESLRSKEAKFVAAWTLKAAAGSGSGSDTICIGYHANNSTDTVDTVLEKNVTVTHSVNLLEDSHNGKLCRLKGIAPLQLGKCNIAGWLLGNPECDPLLPVRSWSYIVETPNSENGICYPGDFIDYEELREQLSSVSSFERFEIFPKESSWPNHNTNGVTAACSHEGKSSFYRNLLWLTEKEGSYPKLKNSYVNKKGKEVLVLWGIHHPPNSKEQQNLYQNENAYVSVVTSNYNRRFTPEIAERPKVRDQAGRMNYYWTLLKPGDTIIFEANGNLIAPMYAFALSRGFGSGIITSNASMHECNTKCQTPLGAINSSLPYQNIHPVTIGECPKYVRSAKLRMVTGLRNNPSIQSRFEDKTHTCPPCPAPELLGGPSVFLFPPKPKDQLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLYSKLTVDKSRWQQGNVFSCSVLHEALHNHYTQKSLSLSPGK

SEQ ID NO: 68 - protein, 510 aa, Homo sapiens
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFRRITLYLKEKKYSPCAWEVVRAEIMRSFSLSTNLQESLRSKEAKFVAAWTLKAAAGSGSGSMHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQKPFEDKTHTCPPCPAPELLGGPSVFLFPPKPKDQLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLYSKLTVDKSRWQQGNVFSCSVLHEALHNHYTQKSLSLSPGK

SEQ ID NO: 69 - protein, 414 aa, Homo sapiens; identical to SEQ ID NO: 60

SEQ ID NO: 70 - protein, 356 aa, Homo sapiens; identical to SEQ ID NO: 34

SEQ ID NO: 71 - protein, 471 aa, Homo sapiens; identical to SEQ ID NO: 35

SEQ ID NO: 72 - protein, 574 aa, Homo sapiens; identical to SEQ ID NO: 36

SEQ ID NO: 73 - protein, 346 aa, Homo sapiens; identical to SEQ ID NO: 37

SEQ ID NO: 74 - protein, 118 aa, Homo sapiens
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSA

SEQ ID NO: 75 - protein, 108 aa, Homo sapiens
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKR

SEQ ID NO: 76 - protein, 1258 aa, Hepatitis B virus
VNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 77 - protein, 670 aa, Hepatitis B virus
VNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRAR

SEQ ID NO: 78 - protein, 223 aa, Hepatitis B virus; identical to SEQ ID NO: 8

SEQ ID NO: 79 - protein, 223 aa, Hepatitis B virus
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKCVNF

SEQ ID NO: 80 - protein, 223 aa, Hepatitis B virus
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNF

SEQ ID NO: 81 - protein, 223 aa, Hepatitis B virus
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNF

SEQ ID NO: 82 - protein, 223 aa, Hepatitis B virus
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 83 - protein, 223 aa, Hepatitis B virus
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVQGFNCYFPLQSYGFQPTNGVGYQPYRVVVLSFELLHAPATVCGPKKSTNLVKNKCVNF

SEQ ID NO: 84 - protein, 223 aa, Hepatitis B virus
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNF

SEQ ID NO: 85 - protein, 150 aa, Mus musculus
SRACPPCAPACKDNHCWGESPEDCQILTGTICTSGCARCKGRLPTDCCHEQCAAGCTGPKHSDCLACLHFNHSGICELHCPALVTYNTDTFESMHNPEGRYTFGASCVTTCPYNYLSTEVGSCTLVCPPNNQEVTAEDGTQRCEKCSKPC

SEQ ID NO: 86 - protein, 121 aa, Mus musculus
GCKKIFGSLAFLPESFDGDPSSGIAPLRPEQLQVFETLEEITGYLYISAWPDSLRDLSVFQNLRIIRGRILHDGAYSLTLQGLGIHSLGLRSLRELGSGLALIHRNAHLCFVHTVPWDQLF

SEQ ID NO: 87 - protein, 134 aa, Mus musculus
VCNSLCAHGHCWGPGPTQCVNCSHFLRGQECVEECRVWKGLPREYVSDKRCLPCHPECQPQNSSETCFGSEADQCAACAHYKDSSSCVARCPSGVKPDLSYMPIWKYPDEEGICQPCPINCTHSCVDLDERGCP

SEQ ID NO: 88 - protein, 150 aa, Homo sapiens
SRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPC

SEQ ID NO: 89 - protein, 117 aa, Homo sapiens
GCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPW

SEQ ID NO: 90 - protein, 133 aa, Homo sapiens
CHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCP

SEQ ID NO: 91 - protein, 508 aa, Hepatitis B virus
SVLRYDDFHTDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVDQRQYGDVFKGDLNPKPQGQRLIEVSVEENHPFTLRAPIQRIYGVRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSTCLYHPNAPQCLSHMNSGCTFTSPHLAQRVASTVYQNCEHADNYTAYCLGISHMEPSFGLILHDGGTTLKFVDTPESLSGLYVFVVYFNGHVEAVAYTVVSTVDHFVNAIEERGFPPTAGQPPATTKPKEITPVNPGTSPLLRY

SEQ ID NO: 92 - protein, 425 aa, Hepatitis B virus
MEAALLVCQYTIQSLIHLTGEDPGFFNVEIPEFPFYPTCNVCTADVNVTINFDVGGKKHQLDLDFGQLTPHTKAVYQPRGAFGGSENATNLFLLELLGAGELALTMRSKKLPINVTTGEEQQVSLESVDVYFQDVFGTMWCHHAEMQNPVYLIPETVPYIKWDNCNSTNITAVVRAQGLDVTLPLSLPTSAQDSNFSVKTQMLGNEIDIECIMEDGEISQVLPGDNKFNITCSGYESHVPSGGILTSTSPVVTPIPGTGYAYSLRLTPRPVSRFLGNNSILYVFYSGNGPKASGGDYCIQSNIVFSDEIPASQDMPTNTTDITYVGDNATYSVPMVTSEDANSPNVTVTAFWAWPNNTETDFKCKWTLTSGTPSGCENISGAFASNRTFDITVSGLGTAPKTLIITRTATNATTTTHKVIFSKAP

SEQ ID NO: 93 - protein, 314 aa, Hepatitis B virus
KYALADPSLKMADPNRFRGKNLPVLDRLTDPPGVKRVYHIQPSLEDPFQPPSIPITVYYAVLERACRSVLLHAPSEAPQIVRGASDEARKHTYNLTIAWYRMGDNCAIPITVMEYTECPYNKSLGVCPIRTQPRWSYYDSFSAVSEDNLGFLMHAPAFETAGTYLRLVKINDWTEITQFILEHRARASCKYALPLRIPPAACLTSKAYQQGVTVDSIGMLPRFIPENQRTVALYSLKIAGWHGPKPPYTSTLLPPELSDTTNATQPELVPEDPEDSALLEDPAGTVSSQIPPNWHIPSIQDVAPHHAPAAPSNP

(19) United States
(12) Patent Application Publication
(10) Pub. No.: US 2025/0228935 A1
(43) Pub. Date: Jul. 17, 2025

(54) CONSTRUCTION AND APPLICATION OF FUSION PROTEIN VACCINE PLATFORM

(71) Applicant: INSTITUTE OF BIOPHYSICS, CHINESE ACADEMY OF SCIENCES, Beijing (CN)

(72) Inventors: Yangxin FU, Beijing (CN); Hua PENG, Beijing (CN); Shiyu SUN, Beijing (CN)

(21) Appl. No.: 19/172,135

(22) Filed: Apr. 7, 2025

Related U.S. Application Data
(62) Division of application No. 18/003,872, filed on Dec. 29, 2022.

(30) Foreign Application Priority Data
Jul. 1, 2020 (CN) 202010623708.8
Mar. 31, 2021 (CN) 202110353488.6

(51) Int. Cl. A61K 39/395; A61K 9/00; A61K 39/00; A61K 39/12; A61K 40/42; A61P 31/12

(52) U.S. Cl. CPC A61K 39/395; A61K 9/0019; A61K 39/12; A61K 40/4204; A61P 31/12; A61K 2039/6056; A61K 2039/6075

(57) ABSTRACT

The present invention relates to the construction and application of a fusion protein vaccine platform. The present invention provides a vaccine, comprising a fusion protein containing an interferon-target antigen-immunoglobulin Fc region (or antibody) and a Th cell helper epitope. The present invention also relates to use of a fusion protein containing an interferon-target antigen-immunoglobulin Fc region (or antibody) and a Th cell helper epitope in the preparation of prophylactic or therapeutic compositions. The vaccine of the present invention can be produced by eukaryotic cell expression systems to prepare wild-type and various mutant antigen vaccines, and vaccination by means of subcutaneous/muscular or nasal or other routes can lead to a strong immune response to a body. The vaccine of the present invention can be used as a prophylactic or therapeutic vaccine.

### SEQUENCE LISTING

The instant application contains a Sequence Listing which has been submitted electronically as XML file format and is hereby incorporated by reference in its entirety. Said XML copy, created on Mar. 11, 2025, is named 0259-IB01US2 Sequence Listing and is 39,604 bytes.

### FIELD OF THE INVENTION

The invention belongs to the field of genetic engineering and biomedical technology, and specifically relates to vaccines, for example, a vaccine comprising a fusion protein containing an interferon-target antigen-immunoglobulin Fc region (antibody) as framework. The vaccine of the present invention can be used as a vaccine platform for preventing hepatitis B virus (HBV) infection and for treating chronic hepatitis B (CHB) infection and HBV-related tumors.

### BACKGROUND OF THE INVENTION

There are about 257 million chronic HBV infections in the world, and about 887,000 people die each year from end-stage liver diseases caused by HBV, including liver failure, liver cirrhosis, and hepatocellular carcinoma^{[1-3]}. About 30% of liver cirrhosis is caused by HBV, and about 40% of hepatocellular carcinoma (HCC) is caused by HBV^{[4]}. HBV infection remains a major public health problem worldwide. However, there is still no effective treatment strategy for chronic hepatitis B. The existing HBV treatment methods mainly include antiviral drugs (nucleoside/nucleotide analogs) and interferon. Although they have certain therapeutic effects, they usually cannot induce an effective immune response, so that HBV infection cannot be completely eliminated; moreover, long-term dosing may lead to significant side effects, and antiviral drugs will also lead to drug resistance. Chronic HBV infection is one of the main diseases that threaten human health. It is imminent to explore effective immunotherapy strategies for chronic hepatitis B. The development of therapeutic vaccines for chronic hepatitis B has very important social and economic significance.

The linkage of an antigen to Fc region of an immunoglobulin will significantly increase the half-life of the antigen, and the Fc region of the immunoglobulin can bind to Fc receptors on the surface of antigen-presenting cells to promote the processing and presenting of the antigen by antigen-presenting cells^{[5-7]}. Type I interferon has many biological activities as an antiviral cytokine, which includes the stimulation of immune cells^{[8]}. IFNα can strongly induce the differentiation and activation of human DC cells^{[9]}. Upon acting on immature DCs, type I interferon can promote the expression of MHC molecules and co-stimulatory molecules on the surface of DCs, such as MHC class I, CD80 and CD86, thereby enhancing the ability of DCs to activate T cells^{[10-12]}. It has been reported that type I interferon can promote the antigen-presenting ability of DCs after infection with vaccinia virus and Lymphocytic ChorioMeningitis Virus (LCMV)^{[13-15]}. In addition, type I interferon can promote the migration of DCs to lymph nodes by up-regulating the expression of chemokine receptors after acting on DCs, thereby promoting the activation of T cells[^{16, 17]}. Recently, more and more studies have shown that type I interferon can be used as an immune adjuvant. The study by Le Bon et al. showed that when mice were immunized with a weak immunogen, type I interferon exhibited a strong immune adjuvant effect in mice and induced long-lasting antibodies and immune memory^{[18]}, the author also found that the main cell populations in which type I interferon exerted its effect were DC cells. At the same time, antibodies are used to targeted deliver vaccines to DCs to stimulate DC activation and cross-presentation functions, which will further enhance the activity and potency of the vaccines.

There is a need for the present invention to provide a vaccine platform that enhances the response to virus antigens.

### SUMMARY OF THE INVENTION

Vaccines are an effective way to prevent and control major outbreaks of infectious diseases. There are various types of vaccines, one of which is protein subunit vaccines. In general, simple protein subunit vaccines generally have poor immunogenicity, which often limits the use of protein subunit vaccines. Therefore, a universal protein subunit vaccine platform is urgently needed. According to the impact of immunoglobulin Fc region and type I interferon on the immune system, the inventors propose a interferon alpha-viral antigen-immunoglobulin Fc region fusion protein vaccine platform to enhance the immune response to virus antigens. The present invention provides a type I interferon-protein antigen-immunoglobulin Fc vaccine platform, wherein the type I interferon can promote antigen-presenting cells to allow maturation and migration so as to better play the role in antigen presentation and T cell activation. On the other hand, the Fc moiety of the vaccine platform can bind to the Fc receptors on the surface of antigen-presenting cells to enhance the uptake of antigens by antigen-presenting cells, thereby further enhancing antigen-presenting cells to function. The present inventors propose that the fusion of Th cell helper epitopes can further enhance the immune response effect of the vaccine of type I interferon-protein antigen-immunoglobulin Fc, and thus the Th cell helper epitope is an important element of the vaccine. The present inventors propose that anti-PD-L1 and other antibodies can be used to replace Fc, and the vaccine can be delivered to DCs to stimulate DC activation and cross-presentation, which will further enhance the activity and potency of the vaccine. As a novel vaccine platform, the vaccine platform of the present invention can be used as a prophylactic and therapeutic vaccine for diseases such as viral infections.

In some embodiments, the present invention provides a vaccine comprising a fusion protein containing an interferon-target antigen-immunoglobulin Fc region (or antibody) (and an additional Th epitope). In some embodiments, the present invention also provides use of the fusion protein containing an interferon-target antigen-immunoglobulin Fc region (or antibody) (and an additional Th epitope) for the preparation of prophylactic or therapeutic compositions or kits (such as medicaments or vaccine compositions or kits). The vaccine of the present invention can be produced by eukaryotic cell expression systems, and inoculated by means of subcutaneous/muscular or intranasal or other immunization routes. For the fusion polypeptide of the present invention, the antibody (Ab for short) as a structural unit is not particularly limited, and may include, for example, a complete antibody or a fragment of antibody, such as an antibody heavy chain and light chain, or a single-chain antibody, and may be antibodies for DC targeting activation, including anti-PD-L1, anti-DEC205, anti-CD80/86 and other antibodies.

In some embodiments, the target antigen described herein is not particularly limited and may be any appropriate antigen. In some embodiments, the target antigens described herein can be, for example, viral antigens.

In some embodiments, the target antigen used in the vaccine of the present invention can be, for example, a mutated target antigen that is different from the wild type. In some embodiments, the target antigen described herein can be, for example, mutants of viral antigens. Herein, the wild-type target antigen refers to viruses or other infectious agents encoded by wild-type genes or immunogenic proteins expressed by tumors (the wild-type gene refers to the prevalent allele in nature, and is often used as a standard control gene in biological experiments). Herein, the mutated target antigen (mutant) refers to mutated viral proteins expressed by mutant virus strains and encoded by mutated gene derived from the wild-type genes. In some embodiments, mutated target antigens may include for example natural point mutation/deletion mutation/addition mutation/truncation, artificial point mutation/deletion mutation/addition mutation/truncation, any combination of natural or artificial mutations, subtypes generated by mutations, wherein the target antigen may be a virus antigen. In some embodiments, the target antigen used in the vaccine of the present invention is a mutated viral antigen. Herein, unless otherwise clearly stated or clearly limited by the context, the target antigen herein generally includes wild-type target antigens and mutant target antigens.

The object of the present invention is to provide a vaccine platform, which consists of an interferon (IFN) and a virus antigen (hepatitis B virus Pres1 antigen, hepatitis B virus surface antigen (HBsAg) antigen or peptide fragment)-immunoglobulin Fc region (or antibody) (and an additional Th epitope). The fusion protein can be a homodimeric or heterodimeric protein. In the case that the fusion protein is in the form of a dimer, the interferon, the target antigen, and the immunoglobulin Fc region (or antibody Ab) as structural units can exist in the first polypeptide chain and/or the second polypeptide chain, and the existence of each structural unit is not particularly limited, for example, they can all exist in one chain, or any one or more structural units can exist in one chain, while other one or more structural units can exist in another chain.

The interferon of the present invention can be selected from type I interferon, type II interferon and type III interferon, such as IFN-α, IFN-β, IFN-γ, IFN-λ1 (IL-29), IFN-λ2 (IL-28a), IFN-λ (IL-28b) and IFN-ω; the IFN can be derived from human or mouse; preferably type I interferon IFN-α (SEQ ID NO. 1, SEQ ID NO. 11, SEQ ID NO. 12).

The immunoglobulin Fc region of the present invention can be selected from the constant region amino acid sequences of IgG1, IgG2, IgG3 and IgG4/or IgM, preferably IgG1 (SEQ ID NO. 2, SEQ ID NO. 13, SEQ ID NO. 14).

The fusion polypeptide of the present invention may also optionally comprise one or more Th cell helper epitopes and/or linking fragments (linkers). For example, when the fusion protein is in the form of a dimer, optionally the fusion protein can also comprises one or more Th cell helper epitopes and/or linking fragments in any one or two chains of the homodimer or heterodimer (i.e. the first polypeptide chain and/or or the second polypeptide chain). As known to those skilled in the art, the various structural units of the fusion protein can be connected by appropriate linking fragments (linkers). The linking fragments that can be used in the vaccine of the present invention are not particularly limited, and can be any suitable peptide fragments known in the art. The linking fragments of each structural unit in the present invention can be flexible polypeptide sequences, and can be linking fragments 1 and 2, for example as shown in the amino acid sequences of SEQ ID NO. 4 and SEQ ID NO. 15.

The N-terminal of the polypeptide sequence composed of each structural unit in the present invention contains a corresponding signal peptide capable of promoting protein secretion, for example as shown in the amino acid sequence of SEQ ID NO. 5.

Preferred antigens described in the present invention include hepatitis B Pres1 antigen, including ad subtype (SEQ ID NO. 6), ay subtype (SEQ ID NO. 16), HBV HBsAg antigen (various subtypes and peptide fragments), including adr subtype (SEQ ID NO. 7), adw subtype (SEQ ID NO. 17), ayw subtype (SEQ ID NO. 18).

The homodimeric protein described in the present invention comprises a first polypeptide and a second polypeptide, and the first polypeptide and the second polypeptide are completely identical. The order of the elements from N-terminal to C-terminal in the first polypeptide and the second polypeptide is IFN-virus antigen (hepatitis B Pres1 antigen or HBsAg antigen)-immunoglobulin Fc region; or a polypeptide containing a Pan epitope. The homodimeric protein of the present invention comprises the a sequences as shown in SEQ ID NO. 8, 19, 22, 25, 26, 28, or 31.

The heterodimer of the present invention comprises a first polypeptide and a second polypeptide, wherein the first polypeptide and the second polypeptide are not identical; the first polypeptide, from the C terminal to the N terminal, is respectively IFN-immunoglobulin Fc region, and comprises an amino acid sequence as shown in SEQ ID NO. 9, 20, 23, 26, 29, or 32; the second polypeptide, from the C terminal to the N terminal, is respectively a virus antigen (Hepatitis B Pres1 antigen)-immunoglobulin Fc region, and comprises an amino acid sequence as shown in SEQ ID NO. 10, 21, 24, 27, 30, or 33.

The present invention also provides a nucleotide sequence encoding the above IFN-virus antigen (hepatitis B Pres1 antigen, HBsAg antigen or peptide)-immunoglobulin Fc vaccine platform.

The present invention also relates to a nucleotide fragment encoding the vaccine platform and fusion protein.

The present invention also relates to a preparation method of the fusion protein or vaccine platform, for example, the preparation method includes the following steps:
(1) An expression vector comprising the gene encoding the fusion protein or vaccine platform is constructed; preferably, the expression vector is a pEE12.4 expression vector;
(2) A host cell comprising the expression vector is constructed by transient transfection; preferably, the host cell is a 293F cell;
(3) The host cells are cultured and cell supernatant is collected;
(4) The fusion protein or vaccine platform is purified by protein A/G affinity chromatography column.

The present invention also includes the application of the vaccine platform; the vaccine platform can be used as a prophylactic vaccine for hepatitis B, a therapeutic vaccine for hepatitis B.

The present invention includes adjuvants used in the vaccine platform, wherein the adjuvants include aluminum adjuvant (Alum), Toll-like receptor 4 activator ligand MPLA, Toll-like receptor 9 ligand, M59, oligodeoxy Nucleotides (CpG-ODN) and Freund's adjuvant.

The present invention includes the clinical use of the vaccine platform as an HBV therapeutic vaccine in combination with hepatitis B virus envelope protein HBsAg vaccine in the treatment of chronic hepatitis B virus infection.

The present invention includes the clinical use of the vaccine platform as an HBV therapeutic vaccine in combination with nucleoside or nucleotide analogues in the treatment of chronic hepatitis B virus infection.

The present invention includes combined application of the vaccine platform as a prophylactic or therapeutic vaccine for HBV in combination with antiviral drugs and other therapies; as a prophylactic or therapeutic vaccine for HBV-related tumors in combination with antiviral and antitumor drugs and therapies.

The present invention comprises multivalent combination vaccine consisting of the vaccine platform and other virus or pathogen or tumor vaccines.

Any fusion protein vaccine comprising the vaccine platform of the present invention can be inoculated with the adenovirus vaccine, mRNA vaccine, inactivated vaccine or DNA vaccine for the same virus in sequence or simultaneously.

The present invention includes the full-length sequence and any truncation sequence of the vaccine platform antigen.

The present invention comprises any possible mutants of said fusion protein vaccine antigen, including natural point mutation/deletion mutation/truncation, any combination of natural sit mutations, subtypes generated by mutations, and mutated sequences comprising artificial point mutation/deletion mutation/truncation constructed for the purpose of enhancing the effect of the vaccine.

The present invention provides a multivalent combination vaccine consisting any vaccine of the present invention as a component of the vaccine and another vaccine of the present invention or other vaccines different from the vaccine of the present invention such as other virus or pathogen or tumor vaccines: for example, any vaccine of the present invention and the adenovirus vaccine or mRNA vaccine or inactivated vaccine or DNA vaccine for the same virus can be inoculated in sequence or simultaneously. As known in the art, in the case of combination use, the vaccines can be prepared as a convenient kit.

The present invention includes but not limited to the following advantages over the prior art:
1. The antigens of the IFN-virus antigen-immunoglobulin Fc (or antibody) vaccine platform provided by the present invention can be varied in various components, and can be virus-specific antigens, which enhances the flexibility of the vaccine platform, as well as the scope of use of the vaccine platform.
2. In the IFN-virus antigen-immunoglobulin Fc (or antibody) vaccine platform provided by the present invention, the interferon (IFN) can enhance the migration and maturation of antigen-presenting cells, and increase the expression of costimulatory factors, thereby promoting the presentation of antigens to T cells; meanwhile, the Fc region (or antibody) in the vaccine platform, on the one hand, increases the molecular weight of the antigen and thus increases the half-life thereof, and on the other hand, the Fc region (or antibody) can bind to Fc receptors on the surface of antigen-presenting cells and promote the processing and presentation of antigens by antigen-presenting cells, thereby promoting the generation of immune responses.
3. The IFN-virus antigen-immunoglobulin Fc (or antibody) vaccine platform provided by the present invention is expressed by eukaryotic HEK293 cell expression system, and the proteins expressed by HEK293 cells are closer to natural protein molecules either in molecular structure or in physical and chemical characteristics and protein modification and biological functions of proteins.
4. The IFN-virus antigen-immunoglobulin Fc (or antibody) vaccine platform provided by the present invention may be in the form of homodimer or heterodimer, and has better choices for different antigens.
5. The IFN-virus antigen-immunoglobulin Fc vaccine platform provided by the present invention can activate DC to enhance DC cross-presentation and generate strong B cell and T cell immune responses by fusing Th cell helper epitopes such as Pan epitopes, using DC targeting antibodies such as anti-PD-L1, and adding various adjuvants to stimulate immune responses.
6. The IFN-virus antigen-immunoglobulin Fc (or antibody) vaccine platform provided by the present invention has a wide range of applications and can be used not only as a prophylactic vaccine, but also as a therapeutic vaccine.
7. The IFN-virus antigen-immunoglobulin Fc (or antibody) vaccine platform provided by the present invention can not only be used alone, but also can be used as a therapeutic vaccine in combination with existing commercial HBsAg vaccines and nucleoside/nucleotide analogues.
8. The vaccine platform provided by the present invention can be used in combination with other virus or pathogen or tumor vaccines to form a multivalent vaccine.
9. Any fusion protein vaccine in the vaccine platform of the present invention can be inoculated together with the adenovirus vaccine, mRNA vaccine, inactivated vaccine or DNA vaccine for the same virus in sequence or simultaneously.
10. The present invention includes the full-length sequence and any truncation sequence of the vaccine platform antigen.
11. Any possible mutants of the vaccine platform antigen provided in the present invention includes natural point mutation/deletion mutation/addition mutation/truncation, any combination of natural point mutations, subtypes generated by mutations, and mutated sequences comprising artificial point mutation/deletion mutation/addition mutation/truncation constructed for the purpose of enhancing the effect of the vaccine.

### Sequences Involved in the Present Invention:

### 1. Unit Component Sequence:

SEQ ID NO. 1:
Amino acid sequence of mouse mIFNα4 (mIFNα)
CDLPHTYNLGNKRALTVLEEMRRLPPLSCLKDRKDFGFPLEKVDN
QQIQKAQAILVLRDLTQQILNLFTSKDLSATWNATLLDSFCNDLH
QQLNDLKACVMQEPPLTQEDSLLAVRTYFHRITVYLRKKKHSLCA
WEVIRAEVWRALSSSTNLLARLSEEKE
SEQ ID NO. 11:
Amino acid sequence of human IFNα2 (hIFNα)
CDLPQTHSLGSRRTLMLLAQMRRISLFSCLKDRHDFGFPQEEFGN
QFQKAETIPVLHEMIQQIFNLFSTKDSSAAWDETLLDKFYTELYQ
QLNDLEACVIQGVGVTETPLMKEDSILAVRKYFQRITLYLKEKKY
SPCAWEVVRAEIMRSFSLSTNLQESLRSKE
SEQ ID. NO. 12:
Amino acid sequence of human mutant IFNα2
(Q124R) (hmIFNα)
CDLPQTHSLGSRRTLMLLAQMRRISLFSCLKDRHDFGFPQEEFGN
QFQKAETIPVLHEMIQQIFNLFSTKDSSAAWDETLLDKFYTELYQ
QLNDLEACVIQGVGVTETPLMKEDSILAVRKYFRRITLYLKEKKY
SPCAWEVVRAEIMRSFSLSTNLQESLRSKE
SEQ ID NO. 2:
Amino acid sequence of human IgG1-Fc
EPKSCDKTHTCPPCPAPELLGGPSVFLFPPKPKDQLMISRTPEVT
CVVVDVSHEDPEVKFNWYVDGVEVHNAKTKPREEQYNSTYRVVSV
LTVLHQDWLNGKEYKCKVSNKALPAPIEKTISKAKGQPREPQVYT
LPPSRDELTKNQVSLTCLVKGFYPSDIAVEWESNGQPENNYKTTP
PVLDSDGSFLYSKLTVDKSRWQQGNVFSCSVLHEALHNHYTQKSL
SLSPGKHV
SEQ ID NO. 13:
Heterodimer Fc-hole
DKTHTCPPCPAPELLGGPSVFLFPPKPKDTLMISRTPEVTCVVVD
VSHEDPEVKFNWYVDGVEVHNAKTKPREEQYNSTYRVVSVLTVLH
QDWLNGKEYKCKVSNKALPAPIEKTISKAKGQPREPQVCTLPPSR
DELTKNQVSLSCAVKGFYPSDIAVEWESNGQPENNYKTTPPVLDS
DGSFFLVSKLTVDKSRWQQGNVFSCSVMHEALHNHYTQKSLSLSP
GK
SEQ ID NO. 14:
Heterodimer Fc-knob
DKTHTCPPCPAPELLGGPSVFLFPPKPKDTLMISRTPEVTCVVVD
VSHEDPEVKFNWYVDGVEVHNAKTKPREEQYNSTYRVVSVLTVLH
QDWLNGKEYKCKVSNKALPAPIEKTISKAKGQPREPQVYTLPPCR
DELTKNQVSLWCLVKGFYPSDIAVEWESNGQPENNYKTTPPVLDS
DGSFFLYSKLTVDKSRWQQGNVFSCSVMHEALHNHYTQKSLSLSP
GK
SEQ ID NO. 3:
Amino acid sequence of Th helper epitope
Pan HLA DR-binding epitope (PADER)
AKFVAAWTLKAAA
SEQ ID NO. 4:
Amino acid sequence of Linker 1:
GGGGSGGGGSGGGGS
SEQ ID NO. 15:
Amino acid sequence of Linker 2:
GSGSGS
SEQ ID NO. 5:
Amino acid sequence of Signal peptide:
MARLCAFLMILVMMSYYWSACSLG
SEQ ID NO. 6:
Amino acid sequence of HBV Pres1 (ad subtype)
MGGWSSKPRKGMGTNLSVPNPLGFFPDHQLDPAFGANSNNPDWDF
NPIKDHWPAANQVGVGAFGPGLTPPHGGILGWSPQAQGILTTVST
IPPPASTNRQSGRQPTPISPPLRDSHPQA
SEQ ID NO. 16:
Amino acid sequence of HBV Pres1 (ay subtype)
MGQNLSTSNPLGFFPDHQLDPAFRANTANPDWDFNPNKDTWPDAN
KVGAGAFGLGFTPPHGGLLGWSPQAQGILQTLPANPPPASTNRQT
GRQPTPLSPPLRNTHPQA
SEQ ID NO. 7:
Amino acid sequence of HBV HBsAg (adr subtype)
MENTTSGFLGPLLVLQAGFFLLTRILTIPQSLDSWWTSLNFLGGA
PTCPGQNSQSPTSNHSPTSCPPICPGYRWMCLRRFIIFLFILLLC
LIFLLVLLDYQGMLPVCPLLPGTSTTSTGPCKTCTIPAQGTSMFP
SCCCTKPSDGNCTCIPIPSSWAFARFLWEWASVRFSWLSLLVPFV
QWFVGLSPTVWLSVIWMMWYWGPSLYNILSPFLPLLPIFFCLWVY
I
SEQ ID NO. 17:
Amino acid sequence of HBV HBsAg (adw subtype)
MENITSGLLGPLLVLQAGFFLLTRILTIPQSLDSWWTSLSFLGEA
PVCLGQNSQSPTRNHSPTSCPPICPGYRWMCLRRFIIFLFILLLC
LIFLLVLLDYQGMLPVCPLIPGSTTTSTGPCKTCTTPAQGNSMFP
SCCCTKPTDGNCTCIPIPSSWAFAKYLWEWASVRFSWLSLLVPFV
QWFVGLSPTVWLSAIWMIWYWGPSLYSIVCPFTPLLQIFCCLWVF
I
SEQ ID NO. 18:
Amino acid sequence of HBV HBsAg
(ayw subtype)
MENITSGFLGPLLVLQAGFFLLTRILTIPQSLDSWWTSLNFLGGT
TVCLGQSSQSPTSNHSPTSCPPTCPGYRWMCLRRFIIFLFILLLC
LIFLLVLLDYQGMLPVCPLIPGSSTTSTGPCRTCMTTAQGTSMYP
SCCCTKPSDGNCTCIPIPSSWAFGKFLWEWASARFSWLSLLVPFV
QWFVGLSPTVWLSVIWMMWYWGPSLYSILSPFLPLLPIFFCLWVY
I

2. Sequences of Murine IFN Vaccine mIFNα-Antigen-Fc:

SEQ ID NO. 8:
Amino acid sequence of mIFNα-Pres1-Fc in
homodimer
CDLPHTYNLGNKRALTVLEEMRRLPPLSCLKDRKDFGFPLEKVDN
QQIQKAQAILVLRDLTQQILNLFTSKDLSATWNATLLDSFCNDLH
QQLNDLKACVMQEPPLTQEDSLLAVRTYFHRITVYLRKKKHSLCA
WEVIRAEVWRALSSSTNLLARLSEEKESGGGGSGGGGSGGGGSGG
GGRTMGQNLSTSNPLGFFPDHQLDPAFRANTANPDWDFNPNKDTW
PDANKVGAGAFGLGFTPPHGGLLGWSPQAQGILQTLPANPPPAST
NRQTGRQPTPLSPPLRNTHPQAFEEPKSCDKTHTCPPCPAPELLG
GPSVFLFPPKPKDQLMISRTPEVTCVVVDVSHEDPEVKFNWYVDG
VEVHNAKTKPREEQYNSTYRVVSVLTVLHQDWLNGKEYKCKVSNK
ALPAPIEKTISKAKGQPREPQVYTLPPSRDELTKNQVSLTCLVKG
FYPSDIAVEWESNGQPENNYKTTPPVLDSDGSFLYSKLTVDKSRW
QQGNVFSCSVLHEALHNHYTQKSLSLSPGKHV
SEQ ID NO. 9:
Amino acid sequence of the first chain
mIFNα-Fc-hole in heterodimer
CDLPHTYNLGNKRALTVLEEMRRLPPLSCLKDRKDFGFPLEKVDN
QQIQKAQAILVLRDLTQQILNLFTSKDLSATWNATLLDSFCNDLHQQLNDLK
ACVMQEPPLTQEDSLLAVRTYFHRITVYLRKKKHSLCAWEVIRAE
VWRALSSSTNLLARLSEEKESGGGGSGGGGSGGGGSGGGGRTDKT
HTCPPCPAPELLGGPSVFLFPPKPKDTLMISRTPEVTCVVVDVSH
EDPEVKFNWYVDGVEVHNAKTKPREEQYNSTYRVVSVLTVLHQDW
LNGKEYKCKVSNKALPAPIEKTISKAKGQPREPQVCTLPPSRDEL
TKNQVSLSCAVKGFYPSDIAVEWESNGQPENNYKTTPPVLDSDGS
FFLVSKLTVDKSRWQQGNVFSCSVMHEALHNHYTQKSLSLSPGK
SEQ ID NO. 10:
Amino acid sequence of the second chain
Pres1-Fc-knob in heterodimer mIFNα-Pres1-Fc
MGQNLSTSNPLGFFPDHQLDPAFRANTANPDWDFNPNKDTWPDAN
KVGAGAFGLGFTPPHGGLLGWSPQAQGILQTLPANPPPASTNRQT
GRQPTPLSPPLRNTHPQAFEDKTHTCPPCPAPELLGGPSVFLFPP
KPKDTLMISRTPEVTCVVVDVSHEDPEVKFNWYVDGVEVHNAKTK
PREEQYNSTYRVVSVLTVLHQDWLNGKEYKCKVSNKALPAPIEKT
ISKAKGQPREPQVYTLPPCRDELTKNQVSLWCLVKGFYPSDIAVE
WESNGQPENNYKTTPPVLDSDGSFFLYSKLTVDKSRWQQGNVFSC
SVMHEALHNHYTQKSLSLSPGK

### 3. Sequences of Murine IFN and Pan Epitope-Containing Vaccine IFNα-Pan-Antigen-Fc:

SEQ ID NO. 19:
Amino acid sequence of mIFNα-Pan-Pres1-Fc
in homodimer
CDLPHTYNLGNKRALTVLEEMRRLPPLSCLKDRKDFGFPLEKVDN
QQIQKAQAILVLRDLTQQILNLFTSKDLSATWNATLLDSFCNDLH
QQLNDLKACVMQEPPLTQEDSLLAVRTYFHRITVYLRKKKHSLCA
WEVIRAEVWRALSSSTNLLARLSEEKEGGGGSGGGGSGGGGSRTA
KFVAAWTLKAAAGSGSGSMGQNLSTSNPLGFFPDHQLDPAFRANT
ANPDWDFNPNKDTWPDANKVGAGAFGLGFTPPHGGLLGWSPQAQG
ILQTLPANPPPASTNRQTGRQPTPLSPPLRNTHPQAFEDKTHTCP
PCPAPELLGGPSVFLFPPKPKDQLMISRTPEVTCVVVDVSHEDPE
VKFNWYVDGVEVHNAKTKPREEQYNSTYRVVSVLTVLHQDWLNGK
EYKCKVSNKALPAPIEKTISKAKGQPREPQVYTLPPSRDELTKNQ
VSLTCLVKGFYPSDIAVEWESNGQPENNYKTTPPVLDSDGSFLYS
KLTVDKSRWQQGNVFSCSVLHEALHNHYTQKSLSLSPGK
SEQ ID NO. 20:
Amino acid sequence of the first chain
mIFNα-Fc-hole in heterodimer
CDLPHTYNLGNKRALTVLEEMRRLPPLSCLKDRKDFGFPLEKVDN
QQIQKAQAILVLRDLTQQILNLFTSKDLSATWNATLLDSFCNDLH
QQLNDLKACVMQEPPLTQEDSLLAVRTYFHRITVYLRKKKHSLCA
WEVIRAEVWRALSSSTNLLARLSEEKESGGGGSGGGGSGGGGSGG
GGRTDKTHTCPPCPAPELLGGPSVFLFPPKPKDTLMISRTPEVTC
VVVDVSHEDPEVKFNWYVDGVEVHNAKTKPREEQYNSTYRVVSVL
TVLHQDWLNGKEYKCKVSNKALPAPIEKTISKAKGQPREPQVCTL
PPSRDELTKNQVSLSCAVKGFYPSDIAVEWESNGQPENNYKTTPP
VLDSDGSFFLVSKLTVDKSRWQQGNVFSCSVMHEALHNHYTQKSL
SLSPGK
SEQ ID NO: 21:
Amino acid sequence of the second chain
Pan-Pres1-Fc-knob in heterodimer
mIFN-Pan-Pres1-Fc
AKFVAAWTLKAAAGSGSGSMGQNLSTSNPLGFFPDHQLDPAFRAN
TANPDWDFNPNKDTWPDANKVGAGAFGLGFTPPHGGLLGWSPQAQ
GILQTLPANPPPASTNRQTGRQPTPLSPPLRNTHPQAFEDKTHTC
PPCPAPELLGGPSVFLFPPKPKDTLMISRTPEVTCVVVDVSHEDP
EVKFNWYVDGVEVHNAKTKPREEQYNSTYRVVSVLTVLHQDWLNG
KEYKCKVSNKALPAPIEKTISKAKGQPREPQVYTLPPCRDELTKN
QVSLWCLVKGFYPSDIAVEWESNGQPENNYKTTPPVLDSDGSFFL
YSKLTVDKSRWQQGNVFSCSVMHEALHNHYTQKSLSLSPGK

4. Sequences of Human IFN Vaccine hIFNα-Antigen-Fc:

SEQ ID NO. 22:
Amino acid sequence of hIFNα-Pres1-Fc
in homodimer
CDLPQTHSLGSRRTLMLLAQMRRISLFSCLKDRHDFGFPQEEFGN
QFQKAETIPVLHEMIQQIFNLFSTKDSSAAWDETLLDKFYTELYQ
QLNDLEACVIQGVGVTETPLMKEDSILAVRKYFQRITLYLKEKKY
SPCAWEVVRAEIMRSFSLSTNLQESLRSKEGGGGSGGGGSGGGGS
RTMGQNLSTSNPLGFFPDHQLDPAFRANTANPDWDFNPNKDTWPD
ANKVGAGAFGLGFTPPHGGLLGWSPQAQGILQTLPANPPPASTNR
QTGRQPTPLSPPLRNTHPQAFEDKTHTCPPCPAPELLGGPSVFLF
PPKPKDQLMISRTPEVTCVVVDVSHEDPEVKFNWYVDGVEVHNAK
TKPREEQYNSTYRVVSVLTVLHQDWLNGKEYKCKVSNKALPAPIE
KTISKAKGQPREPQVYTLPPSRDELTKNQVSLTCLVKGFYPSDIA
VEWESNGQPENNYKTTPPVLDSDGSFLYSKLTVDKSRWQQGNVFS
CSVLHEALHNHYTQKSLSLSPGK
SEQ ID NO. 23:
Amino acid sequence of the first chain
hIFN-Fc-hole in heterodimer
CDLPQTHSLGSRRTLMLLAQMRRISLFSCLKDRHDFGFPQEEFGN
QFQKAETIPVLHEMIQQIFNLFSTKDSSAAWDETLLDKFYTELYQ
QLNDLEACVIQGVGVTETPLMKEDSILAVRKYFQRITLYLKEKKY
SPCAWEVVRAEIMRSFSLSTNLQESLRSKESGGGGSGGGGSGGGG
SGGGGRTDKTHTCPPCPAPELLGGPSVFLFPPKPKDTLMISRTPE
VTCVVVDVSHEDPEVKFNWYVDGVEVHNAKTKPREEQYNSTYRVV
SVLTVLHQDWLNGKEYKCKVSNKALPAPIEKTISKAKGQPREPQV
CTLPPSRDELTKNQVSLSCAVKGFYPSDIAVEWESNGQPENNYKT
TPPVLDSDGSFKLVSKLTVDKSRWQQGNVFSCSVMHEALHNHYTQ
KSLSLSPGK
SEQ ID NO. 24:
Amino acid sequence of the second chain
Pres1-Fc-knob in heterodimer hIFNα-Pres1-Fc
MGQNLSTSNPLGFFPDHQLDPAFRANTANPDWDFNPNKDTWPDAN
KVGAGAFGLGFTPPHGGLLGWSPQAQGILQTLPANPPPASTNRQT
GRQPTPLSPPLRNTHPQAFEDKTHTCPPCPAPELLGGPSVFLFPP
KPKDTLMISRTPEVTCVVVDVSHEDPEVKFNWYVDGVEVHNAKTK
PREEQYNSTYRVVSVLTVLHQDWLNGKEYKCKVSNKALPAPIEKT
ISKAKGQPREPQVCTLPPSRDELTKNQVSLSCAVKGFYPSDIAVE
WESNGQPENNYKTTPPVLDSDGSFFLVSKLTVDKSRWQQGNVFSC
SVMHEALHNHYTQKSLSLSPGK

### 5. Sequences of Human IFN and Pan Epitope-Containing Vaccine IFNα-Pan-Antigen-Fc Sequence:

SEQ ID NO. 25:
Amino acid sequence of hIFNα-Pan-Pres1-Fc
in homodimer
CDLPQTHSLGSRRTLMLLAQMRRISLFSCLKDRHDFGFPQEEFGN
QFQKAETIPVLHEMIQQIFNLFSTKDSSAAWDETLLDKFYTELYQ
QLNDLEACVIQGVGVTETPLMKEDSILAVRKYFQRITLYLKEKKY
SPCAWEVVRAEIMRSFSLSTNLQESLRSKEAKFVAAWTLKAAAGS
GSGSMGQNLSTSNPLGFFPDHQLDPAFRANTANPDWDFNPNKDTW
PDANKVGAGAFGLGFTPPHGGLLGWSPQAQGILQTLPANPPPAST
NRQTGRQPTPLSPPLRNTHPQAFEDKTHTCPPCPAPELLGGPSVF
LFPPKPKDQLMISRTPEVTCVVVDVSHEDPEVKFNWYVDGVEVHN
AKTKPREEQYNSTYRVVSVLTVLHQDWLNGKEYKCKVSNKALPAP
IEKTISKAKGQPREPQVYTLPPSRDELTKNQVSLTCLVKGFYPSD
IAVEWESNGQPENNYKTTPPVLDSDGSFLYSKLTVDKSRWQQGNV
FSCSVLHEALHNHYTQKSLSLSPGK
SEQ ID NO. 26:
Amino acid sequence of the first chain
hIFNα-Fc-hole in heterodimer
CDLPQTHSLGSRRTLMLLAQMRRISLFSCLKDRHDFGFPQEEFGN
QFQKAETIPVLHEMIQQIFNLFSTKDSSAAWDETLLDKFYTELYQ
QLNDLEACVIQGVGVTETPLMKEDSILAVRKYFQRITLYLKEKKY
SPCAWEVVRAEIMRSFSLSTNLQESLRSKESGGGGSGGGGSGGGG
SGGGGRTDKTHTCPPCPAPELLGGPSVFLFPPKPKDTLMISRTPE
VTCVVVDVSHEDPEVKFNWYVDGVEVHNAKTKPREEQYNSTYRVV
SVLTVLHQDWLNGKEYKCKVSNKALPAPIEKTISKAKGQPREPQV
CTLPPSRDELTKNQVSLSCAVKGFYPSDIAVEWESNGQPENNYKT
TPPVLDSDGSFFLVSKLTVDKSRWQQGNVFSCSVMHEALHNHYTQ
KSLSLSPGK
SEQ ID NO. 27:
Amino acid sequence of the second chain
Pan-Pres1-Fc-knob in
heterodimer hIFNα-Pan-Pres1-Fc
AKFVAAWTLKAAAGSGSGSMGQNLSTSNPLGFFPDHQLDPAFRAN
TANPDWDFNPNKDTWPDANKVGAGAFGLGFTPPHGGLLGWSPQAQ
GILQTLPANPPPASTNRQTGRQPTPLSPPLRNTHPQAFEDKTHTC
PPCPAPELLGGPSVFLFPPKPKDTLMISRTPEVTCVVVDVSHEDP
EVKFNWYVDGVEVHNAKTKPREEQYNSTYRVVSVLTVLHQDWLNG
KEYKCKVSNKALPAPIEKTISKAKGQPREPQVYTLPPCRDELTKN
QVSLWCLVKGFYPSDIAVEWESNGQPENNYKTTPPVLDSDGSFFL
YSKLTVDKSRWQQGNVFSCSVMHEALHNHYTQKSLSLSPGK

6. Sequences of Human Mutated IFN Vaccine hmIFNα-Pan-Antigen-Fc:

SEQ ID NO. 28:
Amino acid sequence of hmIFNα-Pres1-Fc
in homodimer
CDLPQTHSLGSRRTLMLLAQMRRISLFSCLKDRHDFGFPQEEFGN
QFQKAETIPVLHEMIQQIFNLFSTKDSSAAWDETLLDKFYTELYQ
QLNDLEACVIQGVGVTETPLMKEDSILAVRKYFRRITLYLKEKKY
SPCAWEVVRAEIMRSFSLSTNLQESLRSKEGGGGSGGGGSGGGGS
RTMGQNLSTSNPLGFFPDHQLDPAFRANTANPDWDFNPNKDTWPD
ANKVGAGAFGLGFTPPHGGLLGWSPQAQGILQTLPANPPPASTNR
QTGRQPTPLSPPLRNTHPQAFEDKTHTCPPCPAPELLGGPSVFLF
PPKPKDQLMISRTPEVTCVVVDVSHEDPEVKFNWYVDGVEVHNAK
TKPREEQYNSTYRVVSVLTVLHQDWLNGKEYKCKVSNKALPAPIE
KTISKAKGQPREPQVYTLPPSRDELTKNQVSLTCLVKGFYPSDIA
VEWESNGQPENNYKTTPPVLDSDGSFLYSKLTVDKSRWQQGNVFS
CSVLHEALHNHYTQKSLSLSPGK
SEQ ID NO. 29:
Amino acid sequence of the first chain
hmIFN-Fc-hole in heterodimer
CDLPQTHSLGSRRTLMLLAQMRRISLFSCLKDRHDFGFPQEEFGN
QFQKAETIPVLHEMIQQIFNLFSTKDSSAAWDETLLDKFYTELYQ
QLNDLEACVIQGVGVTETPLMKEDSILAVRKYFRRITLYLKEKKY
SPCAWEVVRAEIMRSFSLSTNLQESLRSKESGGGGSGGGGSGGGG
SGGGGRTDKTHTCPPCPAPELLGGPSVFLFPPKPKDTLMISRTPE
VTCVVVDVSHEDPEVKFNWYVDGVEVHNAKTKPREEQYNSTYRVV
SVLTVLHQDWLNGKEYKCKVSNKALPAPIEKTISKAKGQPREPQV
CTLPPSRDELTKNQVSLSCAVKGFYPSDIAVEWESNGQPENNYKT
TPPVLDSDGSFFLVSKLTVDKSRWQQGNVFSCSVMHEALHNHYTQ
KSLSLSPGK
SEQ ID NO: 30:
Amino acid sequence of the second chain
Pres1-Fc-knob in heterodimer hmIFNα-Pres1-Fc
MGQNLSTSNPLGFFPDHQLDPAFRANTANPDWDFNPNKDTWPDAN
KVGAGAFGLGFTPPHGGLLGWSPQAQGILQTLPANPPPASTNRQT
GRQPTPLSPPLRNTHPQAFEDKTHTCPPCPAPELLGGPSVFLFPP
KPKDTLMISRTPEVTCVVVDVSHEDPEVKFNWYVDGVEVHNAKTK
PREEQYNSTYRVVSVLTVLHQDWLNGKEYKCKVSNKALPAPIEKT
ISKAKGQPREPQVYTLPPCRDELTKNQVSLWCLVKGFYPSDIAVE
WESNGQPENNYKTTPPVLDSDGSFFLYSKLTVDKSRWQQGNVFSC
SVMHEALHNHYTQKSLSLSPGK

7. Sequences of Human Mutated IFN and Pan Epitope-Containing Vaccine hmIFNα-Pan Epitope-Antigen-Fc:

SEQ ID NO. 31:
Amino acid sequence of hmIFNα-Pan-Pres1-Fc
in homodimer
CDLPQTHSLGSRRTLMLLAQMRRISLFSCLKDRHDFGFPQEEFGN
QFQKAETIPVLHEMIQQIFNLFSTKDSSAAWDETLLDKFYTELYQ
QLNDLEACVIQGVGVTETPLMKEDSILAVRKYFRRITLYLKEKKY
SPCAWEVVRAEIMRSFSLSTNLQESLRSKEAKFVAAWTLKAAAGS
GSGSMGQNLSTSNPLGFFPDHQLDPAFRANTANPDWDFNPNKDTW
PDANKVGAGAFGLGFTPPHGGLLGWSPQAQGILQTLPANPPPAST
NRQTGRQPTPLSPPLRNTHPQAFEDKTHTCPPCPAPELLGGPSVF
LFPPKPKDQLMISRTPEVTCVVVDVSHEDPEVKFNWYVDGVEVHN
AKTKPREEQYNSTYRVVSVLTVLHQDWLNGKEYKCKVSNKALPAP
IEKTISKAKGQPREPQVYTLPPSRDELTKNQVSLTCLVKGFYPSD
IAVEWESNGQPENNYKTTPPVLDSDGSFLYSKLTVDKSRWQQGNV
FSCSVLHEALHNHYTQKSLSLSPGK
SEQ ID NO. 32:
Amino acid sequence of the first chain
hmIFNα4-Fc-hole in heterodimer
CDLPQTHSLGSRRTLMLLAQMRRISLFSCLKDRHDFGFPQEEFGN
QFQKAETIPVLHEMIQQIFNLFSTKDSSAAWDETLLDKFYTELYQ
QLNDLEACVIQGVGVTETPLMKEDSILAVRKYFRRITLYLKEKKY
SPCAWEVVRAEIMRSFSLSTNLQESLRSKESGGGGSGGGGSGGGG
SGGGGRTDKTHTCPPCPAPELLGGPSVFLFPPKPKDTLMISRTPE
VTCVVVDVSHEDPEVKFNWYVDGVEVHNAKTKPREEQYNSTYRVV
SVLTVLHQDWLNGKEYKCKVSNKALPAPIEKTISKAKGQPREPQV
CTLPPSRDELTKNQVSLSCAVKGFYPSDIAVEWESNGQPENNYKT
TPPVLDSDGSFFLVSKLTVDKSRWQQGNVFSCSVMHEALHNHYTQ
KSLSLSPGK
SEQ ID NO. 33:
Amino acid sequence of the second chain
Pan-Pres1-Fc-knob in heterodimer
hmIFNα-Pan-Pres1-Fc
AKFVAAWTLKAAAGSGSGSMGQNLSTSNPLGFFPDHQLDPAFRAN
TANPDWDFNPNKDTWPDANKVGAGAFGLGFTPPHGGLLGWSPQAQ
GILQTLPANPPPASTNRQTGRQPTPLSPPLRNTHPQAFEDKTHTC
PPCPAPELLGGPSVFLFPPKPKDTLMISRTPEVTCVVVDVSHEDP
EVKFNWYVDGVEVHNAKTKPREEQYNSTYRVVSVLTVLHQDWLNG
KEYKCKVSNKALPAPIEKTISKAKGQPREPQVYTLPPCRDELTKN
QVSLWCLVKGFYPSDIAVEWESNGQPENNYKTTPPVLDSDGSFFL
YSKLTVDKSRWQQGNVFSCSVMHEALHNHYTQKSLSLSPGK

### BRIEF DESCRIPTION OF THE DRAWINGS

FIG. 1 was a schematic diagram of the vaccine platform in the form of homodimer, arranged in the order of interferon-linking fragment 1-target antigen-immunoglobulin Fc (or antibody);

FIG. 2 was a schematic diagram of the vaccine platform in the form of heterodimer, according to the combination of interferon-linking fragment 1-IgG1-hole and target antigen-IgG1-knob (or antibody);

FIG. 3 was a schematic diagram of the vaccine platform in the form of heterodimer, according to the combination of interferon-linking fragment 1-IgG1-knob and target protein-IgG1-hole (or antibody);

FIG. 4 was a schematic diagram of the vaccine platform in the form of homodimer, arranged in the order of interferon-linking fragment 1-Th cell helper epitope-linking fragment 2-target antigen-immunoglobulin Fc (or antibody);

FIG. 5 was a schematic diagram of the vaccine platform in the form of heterodimer, according to the combination of interferon-linking fragment 1-IgG1-hole and Th cell helper epitope-linking fragment 2-target antigen-IgG1-knob (or antibody);

FIG. 6 was a schematic diagram of the vaccine platform in the form of heterodimer, according to the combination of interferon-linking fragment 1-IgG1-knob and Th cell helper epitope-linking fragment 2-target antigen-IgG1-hole (or antibody).

FIG. 7 showed the non-denatured protein SDS-PAGE electrophoresis map of Pres1-Fc, and IFN-Pres1-Fc.

FIG. 8 showed that compared with free preS1, the fusion proteins preS1-Fc and IFN-preS1-Fc could significantly enhance the immunity of antigen molecules and promote the production of broad-spectrum neutralizing antibodies. (a) C57/BL6 (n=8/group) mice were subcutaneously immunized with free hepatitis B Pres1, Pres1-Fc, and IFNα-Pres1-Fc proteins, and the level of Pres1-specific antibody in serum was detected by ELISA at specified time. (b) Mice (n=4) stably carrying three HBV genotypes were injected intravenously with serum from mice immunized with IFNα-Pres1-Fc protein, and the changes of Pres1 antigen in serum were detected 12 hours later.

FIG. 9 showed that IFNα-Pres1-Fc could be used as a prophylactic vaccine against hepatitis B. C57/BL6 mice were subcutaneously immunized with free hepatitis B Pres1, Pres1-Fc, and IFNα-Pres1-Fc proteins, and infected with 1×10¹¹ μg of AAV-HBV1.3 virus by tail vein at day 28 after inoculation. (a) Serum Anti-Pres1 levels before virus inoculation and at 1, 2, 3, and 4 weeks after virus inoculation. (b) Serum levels of Pres1 detected at the indicated time points. (c) Serum HBsAg levels detected at weeks 1, 2, 3, and 4 by ELISA. (d) Proportion of HBsAg-positive mice after AAV-HBV1.3 virus inoculation.

FIG. 10 showed IFNα-Pres1-Fc as a therapeutic vaccine for chronic B infection. C57/BL6 mice were infected with 1×10¹¹ μg of AAV-HBV1.3 virus by tail vein injection. After 6 weeks of infection, stable infected mice were selected (n=8/group), and subcutaneously inoculated with recombinant Pres1, IFNα-Pres1-Fc proteins once every two weeks for a total of three times. (a) Detection of Anti-Pres1 antigen in serum; (b) Detection of Pres1 antigen in serum; (c) Detection of HBV-related antigen HBsAg in mouse serum

FIG. 11 showed that Th cell helper epitopes enhanced the antibody response of IFNα-Pres1-Fc vaccine

Compared with IFN-preS1-Fc, the IFN-Pan-preS1-Fc could significantly enhance the immunogenicity of antigen molecules. C57/BL6 (n=8/group) mice were subcutaneously immunized with hepatitis B Pres1, Pres1-Fc, and IFNα-Pres1-Fc proteins without aluminum adjuvant, and the level of Pres1-specific antibody in serum was detected by ELISA at specified time.

FIG. 12 showed IFNα-Pan-Pres1-Fc as a therapeutic vaccine for chronic B infection. C57/BL6 mice were infected with 1×10¹¹ μg of AAV-HBV1.3 virus by tail vein injection. After 6 weeks of infection, stable infected mice were selected (n=8/group), and subcutaneously inoculated with recombinant Pres1, IFNα-Pres1-Fc proteins once every two weeks for a total of three times. (a) Detection of Anti-Pres1 antigen in serum; (b) Detection of Pres1 antigen in serum; (c) Detection of HBV-related antigen HBsAg level in mouse serum; (d) Detection of HBV-DNA level in mouse serum by QPCR.

FIG. 13 showed the combination of IFNα-Pres1-Fc and HBsAg commercial vaccine broke immune tolerance against HBsAg and induced HBsAg-HBsAb serological conversion. HBV Carrier mice were subcutaneously immunized with IFNα-Pres1-Fc and HBsAg commercial vaccine once every two weeks for a total of three times. (a) The level of Pres1 in the serum of HBV Carrier mice, (b) the level of HBsAg, (c) the level of Anti-Pres1 in serum, (d) the level of Anti-HBsAg in serum, (e) the level of HBV-DNA in serum. ***, p<0.001

### DETAILED DESCRIPTION OF THE INVENTION

In order to make the objective, technical solution and advantages of the present invention more clear, the present invention is described in detail below with reference to the examples and the accompanying drawings. The Examples are only illustrative of the present invention and are not intended to limit the scope of the present invention, and the Examples are only a part of the present invention, and do not represent all embodiments of the present invention. The scope of the invention is defined by the appended claims.

### Example 1. Design of Vaccine Platform

The vaccine platform of interferon-target antigen-immunoglobulin Fc (or antibody) consists of three structural units, wherein the first structural unit is interferon, the second structural unit is immunoglobulin Fc region (or antibody), and the third unit is target antigen. In the process of construction, the three structural units could be arbitrarily arranged and combined, and the target antigen could be connected to a Th cell helper epitope through a linker 2. The representative designs were as follows:

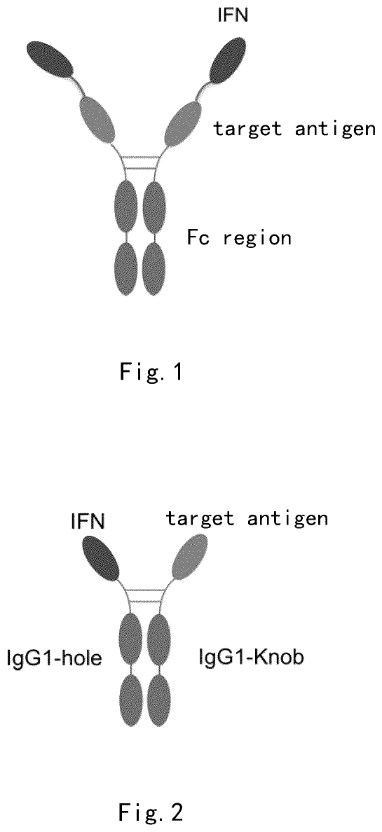

FIG. 1 was a schematic diagram of the vaccine platform in the form of homodimer, arranged in the order of interferon-linking fragment 1-target antigen-immunoglobulin Fc.

FIG. 2 was a schematic diagram of the vaccine platform in the form of heterodimer, according to the combination of interferon-linking fragment 1-IgG1-hole and target antigen-IgG1-knob, respectively.

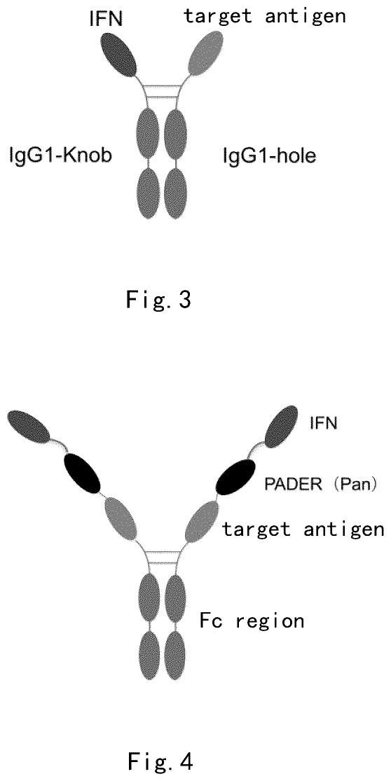

FIG. 3 was a schematic diagram of the vaccine platform in the form of heterodimer, according to the combination of interferon-linking fragment 1-IgG1-knob and target antigen-IgG1-hole.

Next, the inventors tried to connect the target antigen to a cell helper epitope by a linking fragment 2, and then combine it with other two vaccine platform components. The representative designs were as follows:

FIG. 4 was a schematic diagram of the vaccine platform in the form of homodimer, arranged in the order of interferon-linking fragment 1-Th cell helper epitope-linking fragment 2-target antigen-immunoglobulin Fc.

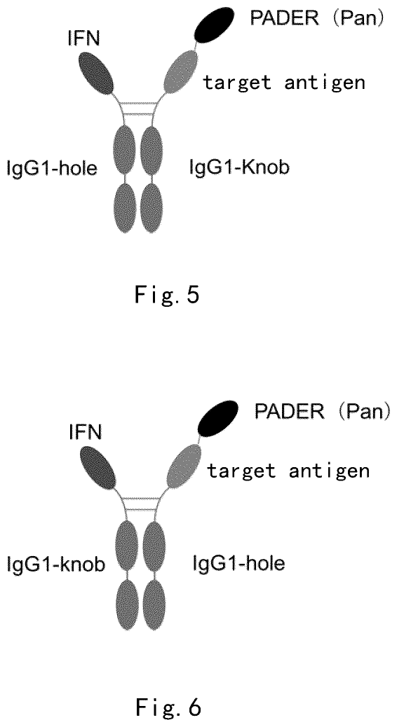

FIG. 5 was a schematic diagram of the vaccine platform in the form of heterodimer, according to the combination of interferon-linking fragment 1-IgG1-hole and Th cell helper epitope-linking fragment 2-target antigen-IgG1-knob.

FIG. 6 was a schematic diagram of the vaccine platform in the form of heterodimer, according to the combination of interferon-linking fragment 1-IgG1-knob and Th cell helper epitope-linking fragment 2-target antigen-IgG1-hole.

### Example 2. Construction, Purification and Production of the Vaccine Platform

The expression and production of the vaccine platform were described by taking hepatitis B virus Pres1 protein homodimer as an example.
1. Vector construction, host cell transfection and induced expression
1.1. The vaccine structural units were constructed on PEE12.4 vector by molecular cloning to obtain a plasmid expressing the fusion protein, which was then transiently transfected into 293F cells, the culture supernatant was collected, and finally the protein of interest was purified by Protein A affinity chromatography.
   Vector Construction (Taking HBV preS1 Antigen as an Example)
(1) PEE12.4-HindII-signal peptide 1-interferon-BsiwI-Pres1-BstbI-hIgG1-EcoRI
(2) PEE12.4-HindII-signal peptide 1-interferon-Bsiwi-PADER-Pres1-hIgG1-EcoRI

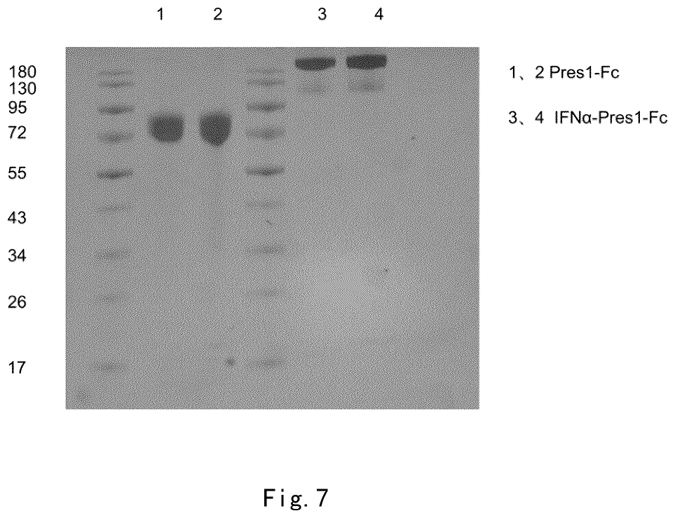

Linkers between each fragment of fusion protein were as follows:
(1) The linker between interferon and Pres1 was linking fragment 1
(2) The linker between interferon and PADER was linking fragment 1, and the linker between PADER and Pres1 was linking fragment 2
1.2. Rapid expression of protein of interest by transient transfection:
(1) Cell thawing: Freestyle 293F cells were frozen in CD OptiCHO™ media (containing 10% DMSO) at a concentration of 3×10⁷ cells/ml. The cells were taken out from liquid nitrogen, and then dissolved quickly in a 37° C. water bath, added into a 15 ml centrifuge tube containing 10 ml OptiCHO™ media, and centrifuged at 1,000 rpm for 5 min. The supernatant was discarded, and the cell pellet was suspended and cultured in 30 ml OptiCHO™ media at 37° C., 8% CO₂, 135 rpm. After 4 days, the cells were subjected to extended culture, and the concentration should not exceed 3×10⁶ cells/ml during the extended culture.
(2) Two days before transfection, the suspension cultured 293F cells were prepared for transient transfection (200 ml) with an inoculum density of 0.6-0.8×10⁶ cells/ml.
(3) Two days later, the suspension of cells to be transfected was counted, and the estimated cell density was 2.5-3.5×10⁶ cells/ml, then the cell suspension was centrifuged at 1,000 rpm for 5 min, and the supernatant was discarded.
(4) Cells were resuspended with 50 ml of fresh Freestyle 293 media, and centrifuged again at 1,000 rpm for 5 min, and the supernatant was discarded.
(5) 293F cells were resuspended with 200 ml Freestyle 293 media.
(6) 600 μg plasmids were diluted with 5 ml of Freestyle 293 media, and filtered by a 0.22 μM filter for sterilization.
(7) 1.8 mg of PEI was diluted with 5 ml of Freestyle 293 media and filtered with a 0.22 μM filter for sterilization. Immediately thereafter, 5 ml of the plasmid and 5 ml of PEI were mixed, and allowed to stand at room temperature for 5 minutes.
(8) The plasmid/PEI mixture was added to the cell suspension, cultured in a 37° C., 8% CO₂, 85 rpm incubator, and meanwhile supplemented with growth factor 50 μg/L LONG™ R3IGF-1.
(9) After 4 hours, 200 ml EX-CELL™ 293 media medium and 2 mM Glutamine were supplemented, and then the cells were continued in culture at 135 rpm.
(10) 24 hours later, 3.8 mM of cell proliferation inhibitor VPA was added; 72 hours later, 40 ml medium D was added, and then the cells were continued in culture; 6-8 days after transfection (the cell survival rate is less than 70%), the supernatant was collected for the next step of purification.
1.3. Collection, purification and electrophoresis verification of fusion protein
2. Purification of protein of interest by using Protein A:
(1) Sample preparation: the cell culture suspension was transferred to a 500 ml centrifuge bucket, and centrifuged at 8,000 rpm for 20 min; precipitate was discarded; and supernatant was filtered by a 0.45 μM filter to remove impurities, and then a final concentration of 0.05% NaN3 was added to prevent bacterial contamination during purification.
(2) Assembly of chromatographic column: An appropriate amount of Protein A Agarose (the amount was calculated by purifying 20 mg of human Fc fusion protein per 1 ml of Protein A) were mixed well, added to the chromatographic column, left at room temperature for about 10 minutes; after separation of Protein A and 20% ethanol solution, the outlet at the bottom was opened to allow the ethanol solution to flow out slowly by gravity.
(3) The chromatographic column was washed and equilibrated with 10 column volumes of distilled water and Binding buffer (20 mM sodium phosphate+0.15M NaCl, pH 7.0), respectively.
(4) The sample was loaded by a constant flow pump at a flow rate of 10 column volumes/hour, and flow-through was collected; and the sample was repeatedly loaded twice.
(5) The column was rinsed with more than 10 column volumes of Binding buffer to remove impurity proteins until no protein was detected in the effluent.
(6) The column was eluted by Elution Buffer (0.1 M Glycine, pH 2.7); eluent was collected in separate tubes, 1 tube for 1 ml eluent; and elution peaks were observed with a protein indicator solution (Bio-Rad protein assay). The collection tubes for the eluted peaks were mixed and added with an appropriate amount of 1 M Tris, pH 9.0 (to adjust the pH to 6-8, which should be more than 0.5 different from the isoelectric point of the purified protein).
(7) The protein of interest was substituted into required buffer by using Zeba desalting spin column or concentrating spin column (please be noted that the pH of the buffer should be adjusted to avoid the isoelectric point of the protein). BSA was used as a standard, and protein concentration was determined by SDS-PAGE electrophoresis and NanoDrop2000.
(8) After elution, the column was washed with 20 column volume of distilled water, and then with 10 column volume of 20% ethanol. Finally, the gel medium should be immersed in ethanol solution and stored at 4° C.
3. The SDS-PAGE electrophoresis map of the protein was shown in FIG. 7.

### Example 3. IFNα-Pres1-Fc, Pres1-Fc could Induce a Stronger Immune Response in Mice than Pres1 Antigen Alone

Materials: C57BL/6 male mice (5-8 weeks old) were purchased from Beijing Vital River Laboratory Animal Technology Co., Ltd.; horseradish peroxidase (HRP)-labeled goat anti-mouse IgG was purchased from Beijing Kangwei Biology Technology Co., Ltd.; 96-well ELISA assay plate was purchased from Corning Costa; ELISA chromogenic solution was purchased from eBioscience; microplate reader SPECTRA max PLUS 384 was purchased from Molecular Company of the United States. The aluminum adjuvant was purchased from SIGMA.

### Methods:

(1) The mice were immunized by Pres1 fusion protein; specially, 80 pmol IFN-Pres1-Fc or 80 pmol Pres1-Fc or Pres1 protein was mixed with aluminum adjuvant and subcutaneously administered to mice. At the designated time points, the serum of the mice was collected by taking blood from the orbit for antibody detection.
(2) The antibody produced by IFNα-Pres1-Fc had extensive neutralizing effect on different genotypes of HBV virus. 5-week-old male C57BL/6 mice were infected with 1×10¹¹ vg of AAV-HBV 1.3 (with HBV genotypes B, C, and D) through tail vein. After 6 weeks, mice with sustained and stable expression of HBV antigen were selected for the test. The selected mice (4 mice/group) were injected intravenously with serum from IFNα-Pres1-Fc immunized mice at 200 ul/mouse. After 12 hours, the serum of the mice was collected, and the changes of the Pres1 antigen in the mice before and after the injection of the antiserum were detected by ELISA.
(3) Anti-Pres1 specific antibody in serum was detected by ELISA. Pres1 (2 μg/ml) coating solution was added to the ELISA plate (Corning 9018) at 50 ul per well, and the plate was coated at 4° C. overnight. The plate was washed once with PBS, 260 uW per well. The plate was blocked with 5% blocking solution (5% FBS) for two hours at 37° C. Serum samples were diluted with PBS (1:10, 1:100, 1:1000, 1:10000), added to the blocked ELISA plate at 50 ul per well and incubated at 37° C. for 1 hour. The plate was washed 5 times with PBST (260 ul for each time), added with enzyme-labeled secondary antibody (enzyme-conjugated anti-mouse IgG-HRP 1:5000 diluted by PBS) at 50 ul per well, and incubated at 37° C. for 1 hour. The plate was washed 5 times with PBST (260 ul for each time), added with substrate TMB 100 ul/well, incubated at room temperature in the dark until color development; 50 ul stop solution (2N H₂SO₄) was added to each well to stop color development, and the plate was read with a microplate reader, at OD450-630.

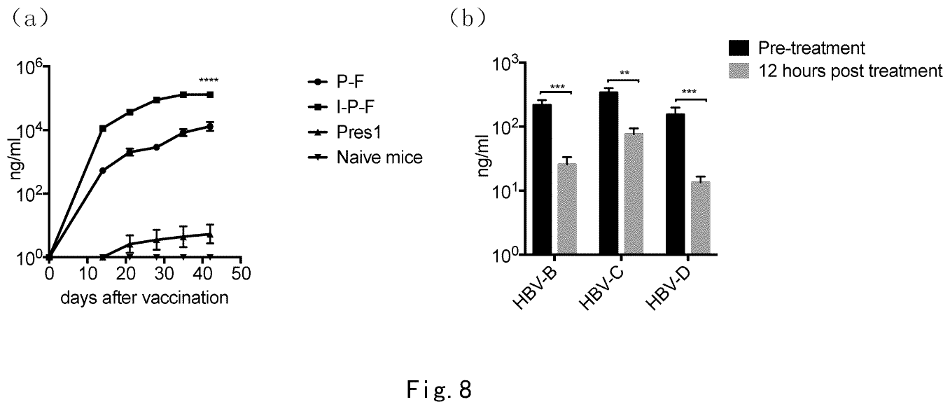

Results: The immunogenicity of free Pres1 was weak, and the immunogenicity was greatly improved when the Pres1 was fused with IFNα and Fc moiety to form IFNα-Pres1-Fc fusion protein, which was shown in FIG. 8(a). The antibody induced by IFNα-Pres1-Fc could produce a wide range of neutralizing effects on different HBV genotypes, as shown in FIG. 8(b).

### Example 4. IFNα-Pres1-Fc could be Used as a Prophylactic Vaccine Against Hepatitis B

Materials: C57BL/6 (6-8 weeks old) male mice were purchased from Beijing Vital River Laboratory Animal Technology Co., Ltd., and HBsAg detection kit was purchased from Shanghai Kehua Bio-Engineering Co., Ltd. AAV-HBV 1.3 virus was purchased from Guangzhou PackGene Biotech Co., Ltd. Other experimental materials were the same as those used in Example 3.

### Methods:

(1) Mice were immunized subcutaneously with 80 pmol of different forms of Pres1 vaccines, including Pres1, Pes1-Fc, and IFNα-Pres1-Fc proteins. At day 28 after immunization, mice serum was collected and mice were infected with 1×10¹¹ vg AAV-HBV 1.3 virus, after that, mouse serum was collected every week for four weeks to detect anti-Pres1 antibody, HBsAg, and Pres1 antigen in the serum. At the third week, peripheral HBV-DNA levels of the mice were detected.
(2) ELISA detection of Pres1-specific antigen in serum. Antigen coating: Pres1 antibody XY007 (4 μg/ml) coating solution was added to the ELISA plate (Corning 9018) at 50 μl per well, and coated overnight at 4° C. The plated was washed once with PBS, 260 μl per well. The plate was blocked with 5% blocking solution (5% FBS) for two hours at 37° C. Serum samples were diluted with PBS (1:10, 1:100), added to the blocked ELISA plate at 50 μl per well (wherein, two duplicate wells were set for each dilution) and incubated at 37° C. for 1 hour. The plate was washed 5 times with PBST (260 μl for each time), added with 50 μl enzyme conjugate (obtained from Kehua HBsAg Detection Kit) per well, and incubated at 37° C. for 1 hour. The plate was washed 5 times with PBST (260 μl for each time), added with substrate TMB 100 μl/well, incubated at room temperature in the dark until color development; 50 μl stop solution (2N H₂SO₄) was added to each well to stop color development, and the plate was read with a microplate reader, at OD450-630.

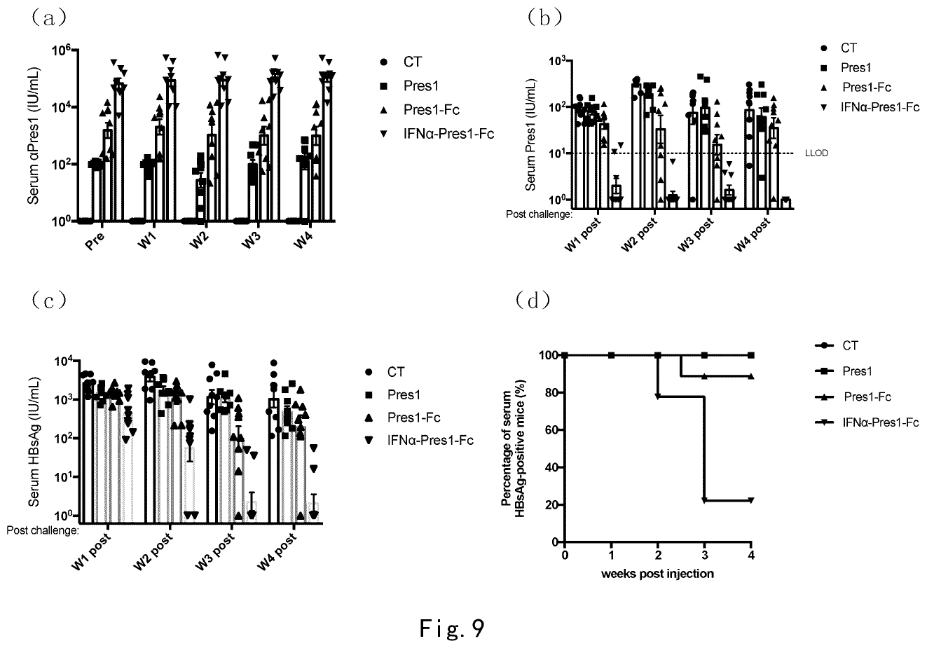

Results: The mice in the IFNα-Pres1-Fc immunized group could produce a high level of Pres1 antibody before inoculation with the virus, and the antibody continued to maintain a high level during the virus infection, as shown in FIG. 9 (a). Compared with the group without protein immunization, IFN-Pres1-Fc vaccine immunization could significantly prevent HBV infection, and the anti-preS1 antibody produced after immunization could quickly and completely clear the preS1 antigen in the serum (FIG. 9(b)), and most of the virus-infected mice in the IFN-Pres1-Fc immunized group were negative for peripheral HBsAg (FIG. 9(c, d)). The above experimental results showed that IFN-Pres1-Fc as a vaccine could effectively prevent HBV infection, as shown in FIG. 9.

### Example 5. IFNα-Pres1-Fc as a Therapeutic Vaccine for Chronic B Infection

Materials: C57BL16 male mice (4 weeks old) were purchased from Beijing Vital River Laboratory Animal Technology Co., Ltd. AAV-HBV 1.3 was purchased from Guangzhou PackGene Biotech Co., Ltd. HBsAg detection kit was purchased from Shanghai Kehua Bio-Engineering Co., Ltd., and other experimental materials were the same as in those in Example 4.

### Methods:

(1) Screening of HBV Carrier mice: 4-week-old HBV C57BL/6 mice were injected with 1×10¹¹ vg AAV-HBV 1.3 virus through tail vein, and HBV antigen HBsAg was detected in 1-6 weeks to screen mice with stable expression of HBsAg which were used as HBV carrier mice for experiments.
(2) The screened mice were subcutaneously injected with 80 pmol of different forms of Pres1 protein, once every two weeks for a total of three immunizations. The mouse serum was collected 14 days after immunization, and then collected once a week, and the levels of anti-Pres1 antibody, HBsAG, and Pres1 antigen in the mouse serum were detected by ELISA. HBV-DNA content in the peripheral blood of the mice was detected after the last blood collection.

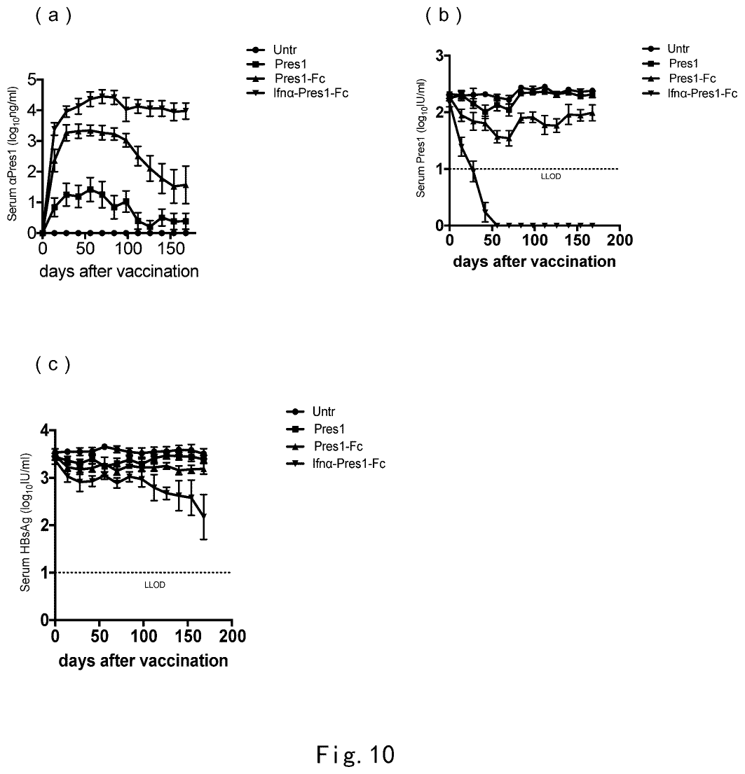

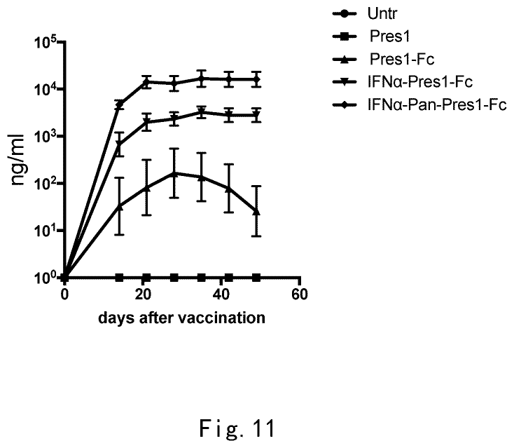

Results: We detected the preS1 antigen in the serum of Carrier mice immunized with IFN-Pres1-Fc vaccine, as well as the changes of Pres1 antibody and HBsAg in the serum. The results showed that after IFNα-Pres1-Fc vaccine immunization, high level of anti-Pres1 antibody in mice was produced, as shown in FIG. 10(a), and the preS1 antigen in the serum could be completely eliminated, as shown in FIG. 10(b). At the same time, HBsAg in the serum also decreased to a certain extent, as shown in FIG. 10(c), while the untreated control group and the Pres1 vaccine immunization group alone had no therapeutic effects, as shown in FIG. 10.

### Example 6. T Cell Helper Epitopes Enhanced the Antibody Response of IFNα-Pres1-Fc Vaccine

Materials: the same as those in Example 3

### Methods:

(1) the mice were immunized by Pres1 fusion proteins, specially, 80 pmol IFN-Pan-Pres1-Fc containing Pan epitope or 80 pmol IFN-Pan-Pres1-Fc, Pres1-Fc, Pres1 protein were subcutaneously inoculated in mice. At the designated time points, the serum of the mice was collected by taking blood from the orbit for antibody detection.
(2) ELISA detection of anti-Pres1 specific antibody in serum, the same as that in Example 3.

Results: Compared with fusion protein vaccines such as IFN-preS1-Fc, the IFN-Pan-preS1-Fc could significantly enhance the immunogenicity of antigen molecules and induce the production of broad-spectrum neutralizing antibodies. C57/BL6 (n=8/group) mice were subcutaneously immunized with hepatitis B Pres1, Pres1-Fc, and IFNα-Pres1-Fc proteins without aluminum adjuvant, and the level of Pres1-specific antibody in serum was detected by ELISA at specified time.

### Example 7. IFNα-Pan-Pres1-Fc as a Therapeutic Vaccine for Chronic B Infection

Materials: C57BL/6 male mice (4 weeks old) were purchased from Beijing Vital River Laboratory Animal Technology Co., Ltd. AAV-HBV 1.3 was purchased from Guangzhou PackGene Biotech Co., Ltd. HBsAg detection kit was purchased from Shanghai Kehua Bio-Engineering Co., Ltd., and other experimental materials were the same as in those in Example 4.

### Methods:

(1) Screening of HBV Carrier mice: 4-week-old HBV C57BL6 mice were injected with 1×10¹¹ vg AAV-HBV 1.3 virus through tail vein, and HBV antigen HBsAg was detected in 1-6 weeks to select mice with stable expression of HBsAg which were used as HBV carrier mice for experiments.
(2) The selected mice were subcutaneously injected with 80 pmol of different forms of Pres1 protein, once every two weeks for a total of three immunizations. The mouse serum was collected 14 days after immunization, and then collected once a week, and the levels of anti-Pres1 antibody, HBsAg, and Pres1 antigen in the mouse serum were detected by ELISA. HBV-DNA content in the peripheral blood of the mice was detected after the last blood collection.

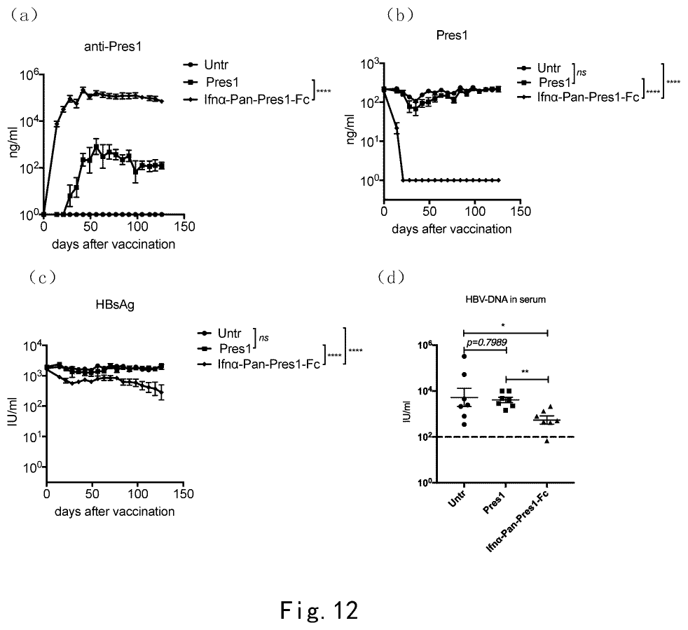

Results: We detected the preS1 antigen in the serum of Carrier mice immunized with IFNα-Pan-Pres1-Fc vaccine, as well as the changes of Pres1 antibody and HBsAg in the serum. The results showed that after IFN-Pan-Pres1-Fc vaccine immunization, the mice produced a high level of anti-Pres1 antibody, as shown in FIG. 12(a). Moreover, the preS1 antigen in the serum could be completely eliminated, as shown in FIG. 12(b), and the HBsAg in the serum also decreased to a certain extent, as shown in FIG. 12(c), while the untreated control group and the Pres1 vaccine immunization group alone had no therapeutic effects. Moreover, the HBV DNA also decreased significantly in the IFNα-Pan-Pres1-Fc immunized group as shown in FIG. 12(d).

### Example 8. The Combination of IFNα-Pan-Pres1-Fc and HBsAg Commercial Vaccine Broke Immune Tolerance Against HBsAg and Induced HBsAg-HBsAb Serological Conversion

Materials: C57BL/6 male mice (4 weeks old) were purchased from Beijing Vital River Laboratory Animal Technology Co., Ltd. AAV-HBV 1.3 was purchased from Guangzhou PackGene Biotech Co., Ltd. HBsAg detection kit was purchased from Shanghai Kehua Bio-Engineering Co., Ltd., and Anti-HBsAg kit was purchased from Beijing Wantai Biological Pharmacy Co., Ltd. Commercial HBsAg vaccine was purchased from Amy Hansen Vaccine (Dalian) Co., Ltd. Other experimental materials were the same as those used in Example 7.

### Methods:

(1) Screening of HBV Carrier mice: 4-week-old HBV C57BL/6 mice were injected with 1×10¹¹ vg AAV-HBV 1.3 virus through tail vein, and HBV antigen HBsAg was detected in 1-6 weeks to select mice with stable expression of HBsAg which were used as HBV carrier mice for experiments.
(2) The selected HBV Carrier mice were immunized with 80 pmol IFNα-pan-Pres1-Fc and 2 μg of commercial HBsAg vaccine at the same time for two consecutive times with an interval of 14 days between each time. The mouse serum was collected 14 days after the first immunization, and the mouse serum was collected every week thereafter, and the changes of anti-Pres1, Pres1, anti-HBsAg, and HBsAg in the serum were detected. And when the mouse serum was collected for the last time, the level of HBV-DNA in the serum was detected.

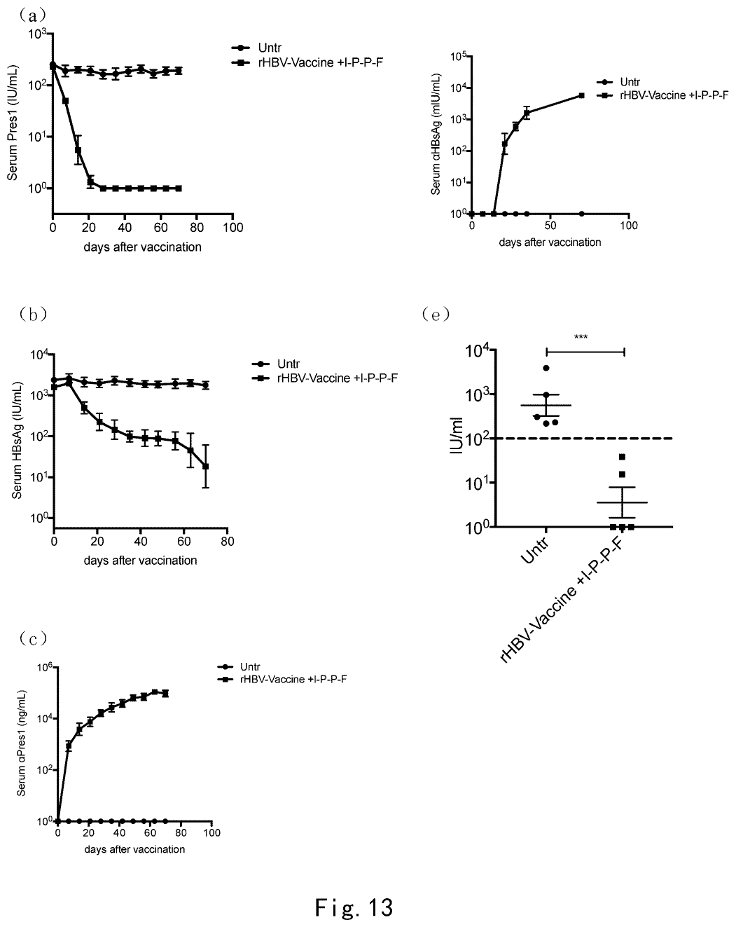

RESULTS: We found that the combination of IFNα-Pan-Pres1-Fc with commercial HBsAg as a strategy for the treatment of chronic hepatitis B could eventually break HBsAg tolerance. The immune response generated in HBV-tolerant mice could completely clear the preS1 antigen in the serum, as shown in FIG. 13(a), and there was a high concentration of Pres1 antibody in the serum (FIG. 13(c)). Excitingly, the IFN-Pan-Pres1-Fc vaccine simultaneously effectively cleared the HBsAg in serum and induced partial serological conversion of HBsAb (FIGS. 13(b) and 4(d)), which were clinically considered as a key indicator of HBV cure. In addition, we detected the expression levels of HBV-related DNA in peripheral blood by fluorescence quantitative real-time PCR. The results showed that, compared with the control group, the immunization by the combination of IFNα-Pan-Pres1-Fc and commercialized HBsAg could finally reduce the level of peripheral HBV DNA (FIG. 13(e)). Based on the above results, we proposed a vaccine strategy for the treatment of chronic hepatitis B by the combination of IFNα-Pan-Pres1-Fc and commercial HBsAg vaccine.

### REFERENCES

[1] WHO W. Global hepatitis report 2017 [J]. Geneva: World Health Organization, 2017,
[2] RAZAVI-SHEARER D, GAMKRELIDZE I, NGUYEN M H, et al. Global prevalence, treatment, and prevention of hepatitis B virus infection in 2016: a modelling study [J]. The lancet Gastroenterology & hepatology, 2018, 3(6): 383-403.
[3] SCHWEITZER A, HORN J, MIKOLAJCZYK R T, et al. Estimations of worldwide prevalence of chronic hepatitis B virus infection: a systematic review of data published between 1965 and 2013 [J]. The Lancet, 2015, 386(10003): 1546-55.
[4] VOS T, ABAJOBIR A A, ABATE K H, et al. Global, regional, and national incidence, prevalence, and years lived with disability for 328 diseases and injuries for 195 countries, 1990-2016: a systematic analysis for the Global Burden of Disease Study 2016 [J]. The Lancet, 2017, 390(10100): 1211-59.
[5] KONTERMANN R E. Strategies for extended serum half-life of protein therapeutics [J]. Current opinion in biotechnology, 2011, 22(6): 868-76.
[6] MEKHAIEL D N. CZAJKOWSKY D M, ANDERSEN J T, et al. Polymeric human Fc-fusion proteins with modified effector functions [J]. Scientific reports, 2011, 1(1): 1-11.
[7] ROOPENIAN D C, AKILESH S. FcRn: the neonatal Fc receptor comes of age [J]. Nature reviews immunology, 2007, 7(9): 715-25.
[8] VILCEK J. Fifty years of interferon research: aiming at a moving target [J]. Immunity, 2006, 25(3): 343-8.
[9] BRACCI L, LA SORSA V, BELARDELLI F, et al. Type I interferons as vaccine adjuvants against infectious diseases and cancer [J]. Expert review of vaccines, 2008, 7(3): 373-81.
[10] HAHM B, TRIFILO M J, ZUNIGA E I, et al. Viruses evade the immune system through type I interferon-mediated STAT2-dependent, but STAT1-independent, signaling [J]. Immunity, 2005, 22(2): 247-57.
[11] ITO T, AMAKAWA R, INABA M, et al. Differential regulation of human blood dendritic cell subsets by IFNs [J]. The Journal of Immunology, 2001, 166(5): 2961-9.
[12] MONTOYA M, SCHIAVONI G, MATTEI F, et al. Type I interferons produced by dendritic cells promote their phenotypic and functional activation [J]. Blood, 2002, 99(9): 3263-71.
[13] LE BON A, ETCHART N, ROSSMANN C, et al. Cross-priming of CD8+ T cells stimulated by virus-induced type I interferon [J]. Nature immunology, 2003, 4(10): 1009-15.
[14] LE BON A, DURAND V, KAMPHUIS E, et al. Direct stimulation of T cells by type I IFN enhances the CD8+ T cell response during cross-priming [J]. The Journal of Immunology, 2006, 176(8): 4682-9.
[15] SPADARO F, LAPENTA C, DONATI S, et al. IFN-α enhances cross-presentation in human dendritic cells by modulating antigen survival, endocytic routing, and processing [J]. Blood, The Journal of the American Society of Hematology, 2012, 119(6): 1407-17.
[16] PARLATO S, SANTINI S M, LAPENTA C, et al. Expression of CCR-7, MIP-3β, and Th-1 chemokines in type I IFN-induced monocyte-derived dendritic cells: importance for the rapid acquisition of potent migratory and functional activities [J]. Blood, The Journal of the American Society of Hematology, 2001, 98(10): 3022-9.
[17] ROUZAUT A, GARASA S, TEUEIRA A, et al. Dendritic cells adhere to and transmigrate across lymphatic endothelium in response to IFN-α [J]. European journal of immunology, 2010, 40(11): 3054-63.
[18] LE BON A, SCHIAVONI G, D'AGOSTINO G, et al. Type I interferons potently enhance humoral immunity and can promote isotype switching by stimulating dendritic cells in vivo [J]. Immunity, 2001, 14(4): 461-70.

I/We claim:

1. A vaccine, which comprises a fusion protein containing an interferon, a target antigen, and an immunoglobulin Fc region, as first structural unit, second structural unit, and third structural unit, respectively,
wherein the interferon is the first structural unit, which is shown in the amino acid sequence of SEQ ID NO. 1, SEQ ID NO. 11, or SEQ ID NO. 12,
wherein the immunoglobulin Fc region is the second structural unit,
wherein the target antigen is the third structural unit, which is HBV Pres1 antigen, and
wherein the fusion protein further contains one or more Th cell helper epitope(s) and linking fragments.

2. The vaccine of claim 1, wherein the fusion protein is a homodimer fusion protein comprising a first polypeptide chain and a second polypeptide chain, wherein the first polypeptide chain and the second polypeptide chain are identical, and each comprises, from N-terminal to C-terminal, the interferon, the Th cell helper epitope(s), the target antigen, and the immunoglobulin Fc region.

3. The vaccine of claim 1, wherein the immunoglobulin Fc region is selected from Fc region of IgG1, IgG2, IgG3, IgG4 and IgM, preferably Fc region of IgG1.

4. The vaccine of claim 1, wherein the target antigen is HBV Pres1 antigen shown in the amino acid sequence of SEQ ID NO. 6, SEQ ID NO. 7, SEQ ID NO. 16, SEQ ID NO. 17, or SEQ ID NO. 18.

5. The vaccine of claim 1, wherein the amino acid sequence of the Th cell helper epitope is shown in SEQ ID NO. 3.

6. The vaccine of claim 1, wherein the fusion protein contains a linking fragment between each structural unit, and the linking fragment is a flexible polypeptide sequence selected from the amino acid sequences of SEQ ID NO. 4 and SEQ ID NO. 15.

7. A nucleic acid molecule encoding the fusion protein in the vaccine of claim 1.

8. An expression vector comprising the nucleic acid molecule of claim 7.

9. A host cell, such as an eukaryotic cell, comprising the nucleic acid molecule of claim 7.

10. A host cell, such as an eukaryotic cell, comprising the expression vector of claim 8.

11. A method of preventing or treating HBV in a subject, comprising administering to the subject the fusion protein in the vaccine defined in claim 1.

12. The method of claim 11, wherein the fusion protein is in a composition or kit.

13. The method of claim 12, wherein the composition or kit is used as a prophylactic or therapeutic vaccine for hepatitis B.

14. The vaccine of claim 1, wherein the vaccine can be inoculated by intramuscular, intravenous, transdermal, subcutaneous or nasal or other immunization routes, wherein the vaccine, the composition or the kit can also comprise an adjuvant, and the adjuvant can comprise aluminum adjuvant (Alum), Toll-like receptor 4 activator ligand MPLA, Toll-like receptor 9 ligand, oligodeoxynucleotide (CpG-ODN), MF59 and Freund's adjuvant.

15. The method of claim 11, wherein the vaccine can be inoculated by intramuscular, intravenous, transdermal, subcutaneous or nasal or other immunization routes, wherein the vaccine, the composition or the kit can also comprise an adjuvant, and the adjuvant can comprise aluminum adjuvant (Alum), Toll-like receptor 4 activator ligand MPLA, Toll-like receptor 9 ligand, oligodeoxynucleotide (CpG-ODN), MF59 and Freund's adjuvant.

16. The vaccine of claim 1, wherein the vaccine can be used in combination with other prophylactic or therapeutic therapies; for example, the vaccine can be hepatitis B therapeutic vaccine, which can be used in combination with another prophylactic or therapeutic hepatitis B therapy, for example, the hepatitis B therapeutic vaccine can be used in combination with hepatitis B virus envelope protein HBsAg vaccine, for example for the treatment of chronic hepatitis B virus infection, for example, the hepatitis B therapeutic vaccine can be combined with nucleoside or nucleotide analogues, for example for the treatment of chronic hepatitis B virus infection, for example, the vaccine can be combined with other vaccines for viruses or pathogens or tumors to form a multivalent vaccine, for example, the vaccine and an adenovirus vaccine or mRNA vaccine or inactivated vaccine or DNA vaccine for the same virus are inoculated in sequence or at the same time.

17. The method of claim 11, wherein the vaccine can be used in combination with other prophylactic or therapeutic therapies; for example, the vaccine can be hepatitis B therapeutic vaccine, which can be used in combination with another prophylactic or therapeutic hepatitis B therapy, for example, the hepatitis B therapeutic vaccine can be used in combination with hepatitis B virus envelope protein HBsAg vaccine, for example for the treatment of chronic hepatitis B virus infection, for example, the hepatitis B therapeutic vaccine can be combined with nucleoside or nucleotide analogues, for example for the treatment of chronic hepatitis B virus infection, for example, the vaccine can be combined with other vaccines for viruses or pathogens or tumors to form a multivalent vaccine, for example, the vaccine and an adenovirus vaccine or mRNA vaccine or inactivated vaccine or DNA vaccine for the same virus are inoculated in sequence or at the same time.

18. A method of preventing or treating hepatitis B virus infection in a subject comprising administering to the subject the fusion protein in claim 1.
